SEQUENCE LISTING (30 sequences)

SEQ ID NO: 1 - RNA, 21 nt, Artificial Sequence
  Artificially synthesized NCT RNAi sense 1
gagagccgcuggaaagauauu

SEQ ID NO: 2 - RNA, 21 nt, Artificial Sequence
  Artificially synthesized NCT RNAi antisense 1
uaucuuuccagcggcucucuu

SEQ ID NO: 3 - RNA, 21 nt, Artificial Sequence
  Artificially synthesized NCT RNAi sense 2
ccacuuauguuguacaguauu

SEQ ID NO: 4 - RNA, 21 nt, Artificial Sequence
  Artificially synthesized NCT RNAi antisense 2
uacuguacaacauaagugguu

SEQ ID NO: 5 - RNA, 21 nt, Artificial Sequence
  Artificially synthesized NCT RNAi sense 3
gcaccaaccuucccacuauuu

SEQ ID NO: 6 - RNA, 21 nt, Artificial Sequence
  Artificially synthesized NCT RNAi antisense 3
auagugggaagguuggugcuu

SEQ ID NO: 7 - RNA, 21 nt, Artificial Sequence
  Artificially synthesized NCT RNAi sense 4
acaaggaucuguaugaguauu

SEQ ID NO: 8 - RNA, 21 nt, Artificial Sequence
  Artificially synthesized NCT RNAi antisense
uacucauacagauccuuguuu

SEQ ID NO: 9 - DNA, 20 bp, Artificial Sequence
  Artificially synthesized Psen2F Primer
ctgcccaggagagaaatgag

SEQ ID NO: 10 - DNA, 20 bp, Artificial Sequence
  Artificially synthesized Psen2R primer
cagtcaagggaggctcaaag

SEQ ID NO: 11 - DNA, 20 bp, Artificial Sequence
  Artificially synthesized Jagged-1F primer
ctcctgtcgggatttggtta

SEQ ID NO: 12 - DNA, 20 bp, Artificial Sequence
  Artificially synthesized JG1R primer
ccacagacgttggaggaaat

SEQ ID NO: 13 - DNA, 20 bp, Artificial Sequence
  Artificially synthesized JG2F primer
gtcaaggtggagacggttgt

SEQ ID NO: 14 - DNA, 20 bp, Artificial Sequence
  Artificially synthesized JG2R primer
atcctcgtcctcctcatcct

SEQ ID NO: 15 - DNA, 20 bp, Artificial Sequence
  Artificially synthesized NT1F primer
gcaacagctccttccacttc

SEQ ID NO: 16 - DNA, 20 bp, Artificial Sequence
  Artificially synthesized NT1R primer
ccacgaagaacagaagcaca

SEQ ID NO: 17 - DNA, 20 bp, Artificial Sequence
  Artificially synthesized NT2F primer
atgactgccctaaccacagg

SEQ ID NO: 18 - DNA, 20 bp, Artificial Sequence
  Artificially synthesized NT2R primer
ctggagtacaggaggcgaag

SEQ ID NO: 19 - DNA, 20 bp, Artificial Sequence
  Artificially synthesized NT3F primer
gtgtgtgtcaatggctggac

SEQ ID NO: 20 - DNA, 20 bp, Artificial Sequence
  Artificially synthesized NT3R primer
cgatagagcactcgtccaca

SEQ ID NO: 21 - DNA, 20 bp, Artificial Sequence
  Artificially synthesized NT4F primer
ggcttctactccgcttcctt

SEQ ID NO: 22 - DNA, 20 bp, Artificial Sequence
  Artificially synthesized NT4R primer
caacttctgcctttggcttc

SEQ ID NO: 23 - DNA, 20 bp, Artificial Sequence
  Artificially synthesized PSENF primer
ggtccacttcgtatgctggt

SEQ ID NO: 24 - DNA, 20 bp, Artificial Sequence
  Artificially synthesized PSENR primer
gcgaggatactgctggaaag

SEQ ID NO: 25 - DNA, 20 bp, Artificial Sequence
  Artificially synthesized PEN2F primer
ttccttgtcccagcctacac

SEQ ID NO: 26 - DNA, 20 bp, Artificial Sequence
  Artificially synthesized PEN2R primer
ggtcctttattgggggatgt

SEQ ID NO: 27 - DNA, 20 bp, Artificial Sequence
  Artificially synthesized APH1F primer
ccgctttgcctactacaagc

SEQ ID NO: 28 - DNA, 20 bp, Artificial Sequence
  Artificially synthesized APH1R primer
ccaaaaggtatggagcagga

SEQ ID NO: 29 - DNA, 20 bp, Artificial Sequence
  Artificially synthesized NicF primer
caaagcaccttcagcatcaa

SEQ ID NO: 30 - DNA, 20 bp, Artificial Sequence
  Artificially synthesized NicR primer
cgagctgccaatgtagtcaa

(19) United States
(12) Patent Application Publication
(10) Pub. No.: US 2010/0266503 A1
(43) Pub. Date: Oct. 21, 2010

(54) Breast Cancer Methods, Medicaments and Agents

(76) Inventors: Martin Slade, London (GB); Raoul Charles Coombes, London (GB)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/594,668

(22) PCT Filed: Apr. 7, 2008
(86) PCT No.: PCT/GB08/01223

§ 371 Date: Jun. 25, 2010

(30) Foreign Application Priority Data
Apr. 5, 2007 (GB) 0706658.2

(51) Int. Cl. A61K 49/00; A61K 31/7088; C07K 16/18; C07K 5/06; A61K 31/275; C12Q 1/37; C12Q 1/68

(52) U.S. Cl. 424/9.2; 514/44 A; 530/389.1; 562/442; 514/523; 435/23; 435/6

(57) ABSTRACT

A method of combating breast cancer in a mammalian individual, the method comprising administering to the individual an agent which reduces the function of the gamma secretase (GS) complex.

The invention relates to methods, medicaments and agents for use in relation to combating breast cancer, inhibiting breast cancer cell growth and in the diagnosis and prognosis of breast cancer.

The listing or discussion of a prior-published document in this specification should not necessarily be taken as an acknowledgement that the document is part of the state of the art or is common general knowledge.

Breast cancer is one of the most frequent and deadly cancers. It is the most commonly diagnosed cancer in women, accounting for 23% of cases (Parkin of al., Global cancer statistics, 2002. CA Cancer J Clin 2005; 55:74-108). There were over 200,000 cases diagnosed in the US alone in 2005, and the incidence of new cases continues to rise worldwide with an estimated 1.45 million new cases globally in 2010. Improvements in breast cancer treatment over recent decades, including the use of screening programs and postoperative adjuvant systemic therapies (hormone therapy and chemotherapy), have reduced overall mortality from the disease. However, despite these improvements, 30-40% of women will still either be diagnosed with metastatic cancer or develop metastases and eventually die from their disease.

Breast cancer is caused by an accumulation of molecular alterations which lead to uncontrolled cell proliferation, genetic instability and the acquisition of an increasingly invasive and resistant phenotype. Having a combinatorial origin, together with the variability of malignant cells and host background, gives rise to various subgroups of molecularly distinct mammary tumours, each with a particular phenotype and clinical outcome.

Research efforts have successfully unravelled some of the mechanisms underlining breast cancer and have identified key genes such as ERBB2, TP53, CCND1, BRCA1 and BRCA2. Moreover, a range of signalling pathways has also been implicated in breast cancer pathogenesis. For example, the MAPK/ERK signalling pathway has been shown to be extremely important in breast cancer cell proliferation and survival. The downstream cascade of this pathway is triggered upon activation of various membrane bound receptors. Growth factor receptors EGFR/ErbB1, as well as HER/ErbB2 execute their function in this manner. The role of EGFR and HER2 receptors is well known in breast cancer as is their contribution to disease progression. More recently, studies have been carried out using tyrosine kinase inhibitors, such as Gefitinib, targeting EGFR, Lapatanib, targeting HER2/EGFR and Herceptin, targeting HER2.

Although this research has led to the development and marketing of new drugs, many patients do not respond to these drugs or indeed become resistant to them. Furthermore, the heterogeneity of breast cancers restricts certain diagnostic or therapeutic strategies to specific breast cancer subtypes such that there is no standardised medication available. Additionally, current cytotoxic drugs do not differentiate between cancerous and normal cells, and so frequently cause adverse secondary effects.

It is apparent therefore, that there remains a significant area of unmet medical need with urgent demand for the development of novel therapeutic agents against breast cancer, that are both effective against several subtypes and which are specific for cancerous cells.

The gamma secretase (GS) complex is an intramembrane aspartyl protease complex that catalyses the intramembrane cleavage of many type I membrane proteins in the process of regulated intramembrane proteolysis. It is composed of four core components: presenilin (PS), anterior pharynx defective (APH-1), presenilin enhancer protein (PEN-2) and nicastrin (NCT), each of which is synthesised as an immature holoprotein in the endoplasmic reticulum.

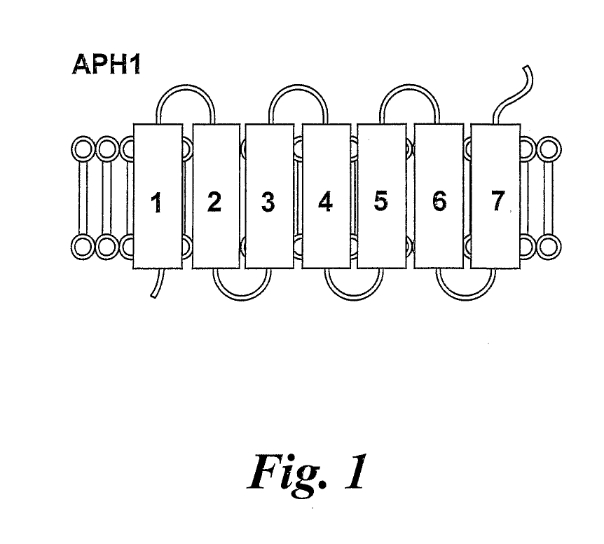

PS has two isoforms: PS1 and PS2, and is synthesised as a 50 KDa holoprotein, undergoing endoproteolysis to produce N and C termini (FIG. 1). PS constitutes the catalytic component of the complex, providing two critical aspartyl residues and is also involved in chaperoning NCT through the Golgi complex. Over a 100 mutations associated with Alzheimer's disease have been documented in PS1 while 6 have been defined in PS2.

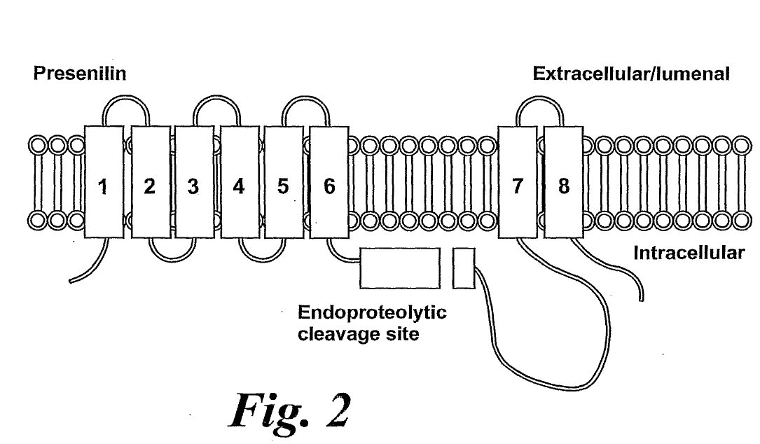

APH-1 has two isoforms: APH1a and APH1b, of which APH1a is the most abundant. It plays a structural role in the assembly of the GS complex, having a membrane imbedded position (FIG. 2).

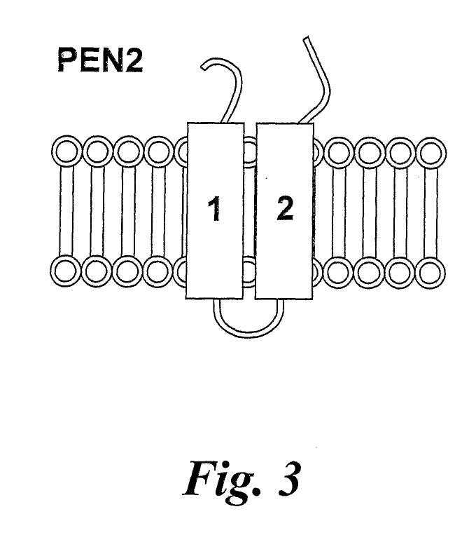

PEN-2 binds to PS at the proximal two thirds of its transmembrane domain where it initiates its endoproteolysis and increases the enzymatic capacity of PS by 70%. Like APH-1, it also plays a structural role in GS assembly owing to its membrane imbedded position (FIG. 3).

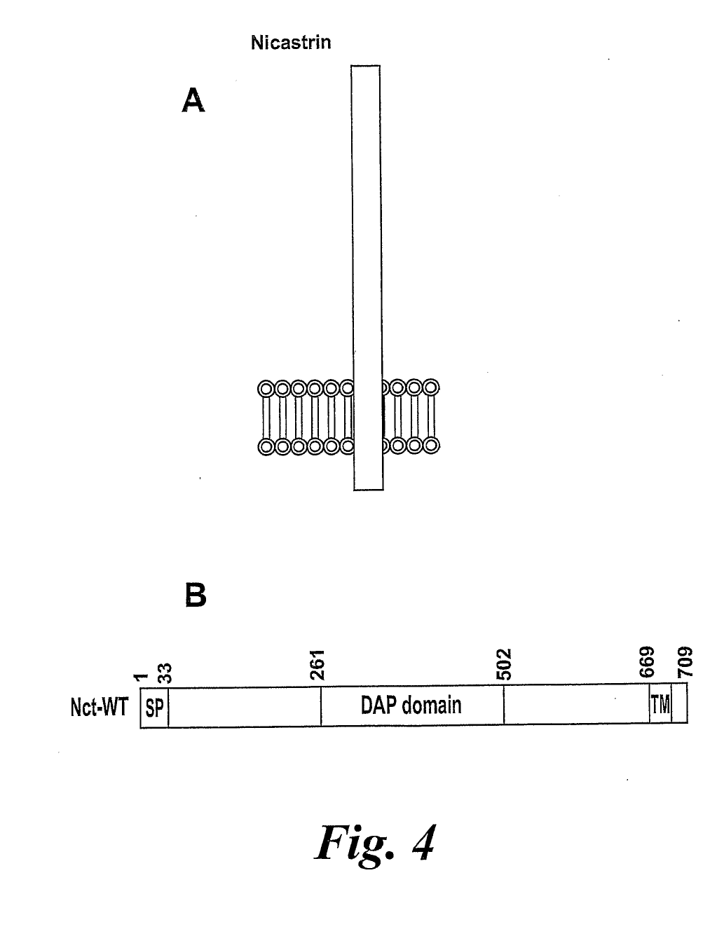

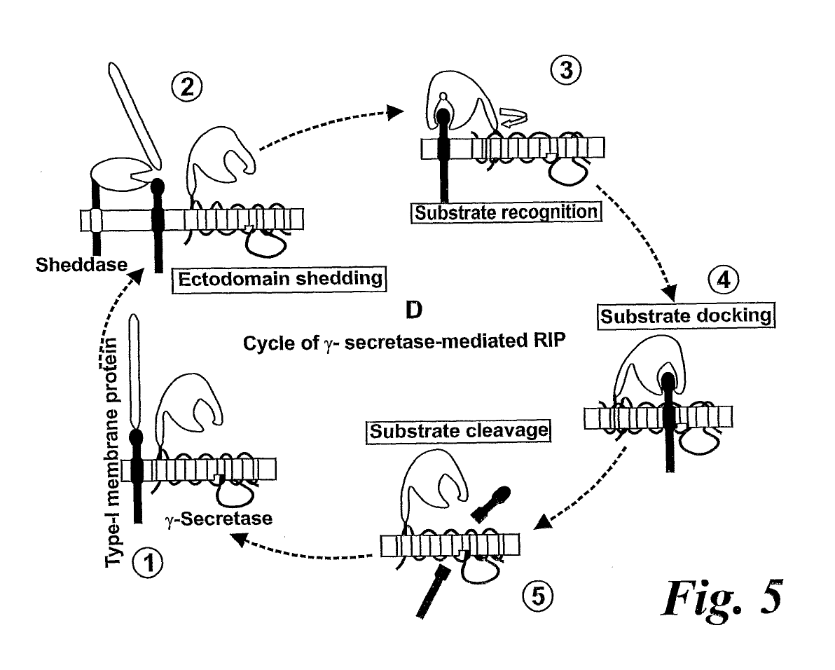

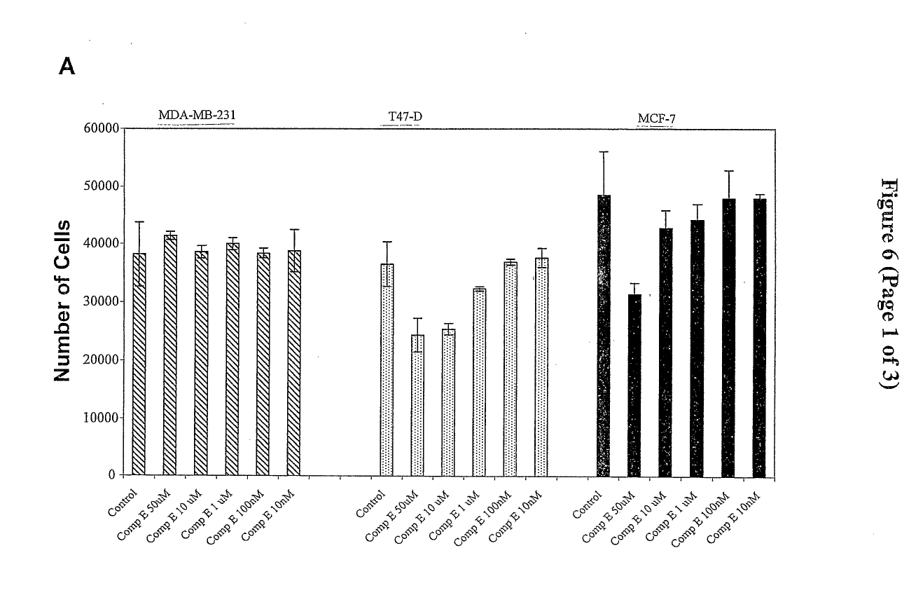

NCT has one transmembrane region as shown in FIG. 4. It is first translated and held in the ER prior to glycosylation and transport through the Golgi complex for maturation. NCT knockouts are embryonically lethal in mice with functional studies revealing roles for NCT in substrate binding and in the assembly of the GS complex. For example, experiments conducted in HEK293 (human embryonic kidney) cells demonstrate that mutation of 28 amino acids in the NCT extracellular DAP domain prevents substrate binding. More specifically, mutating the highly conserved Glu333 residue to Ala333 has the same effect. The DAP domain of NCT is therefore considered essential for substrate binding (FIG. 5). Furthermore, point mutations of the free alpha amino groups of substrates resulted in lack of cleavage, suggesting a direct interaction between NCT and the free alpha amino substrate groups. Indeed, NCT recognises short amino terminus stubs of substrate ectodomains and induces small modifications in them. The juxtamembrane portion of the NCT transmembrane domain has been shown to be necessary for coupling with other enzyme protein members. Mutations (S632A and W648A) performed in HEK293 cells and MEF (mouse embryonic fibroblast) cells, abolished NCT maturation and co-immunoprecipitation assays of NCT with other components of the GS complex show dramatically decreased interactions (De Stooper et al., 2005; Walker et al., 2006; Shah et al., 2005).

The GS complex undergoes a step wise assembly in the endoplasmic reticulum. NCT and APH-1 first form a dimer which is joined by PEN2. PS is then incorporated and becomes activated by PEN2, a step necessary for complex maturation. Maturation is achieved by NCT glycosylation and conformational changes such that the complex increases its molecular weight and becomes resistant to trypsin digestion respectively. The mature complex is exported to the plasma membrane to exert its function (Capell et al., 2005; Hu et al., 2003).

The role of the complex in cleaving the amyloid precursor protein, producing the neurotoxic amyloid β-peptide which is the principle component of Alzheimer's disease plaques, is well known (De Stooper et al., 2005; Capell et al., 2005). In addition, the complex has over 30 recognised substrates including APP, Notch 1-4, Notch ligands Jagged and Delta like 1, 2 and 4, cadherins, syndecans, p75 neurotrophin receptor, ErbB4, CD44 and nectin-1α (Table 1) (Nyborg et al., 2006).

TABLE 1
Substrates of the GS Complex

| Involved In: | Substrate | Role |
|---|---|---|
| Cell | CD44 | Proliferation, differentiation, migration, chaperone of growth factors |
| Adhesion | E-Cadherin | Cell matrix formation |
| | N-Cadherin | Cell adhesion |
| | Nectin-1a | Adherens junction formation |
| | Syndecan 3 | Cell surface proteoglycan co-receptor |
| General cellular | Ire1a | Endoplasmic reticulum protein, sensor in cellular response to stress |
| processes | LRP | Endocytic receptor |
| | VGSC β1-4 | Voltage gates sodium channels |
| Apoptosis | NRADD | Neurotrophin receptor alike death domain |
| | p75 | Neurotrophin co-receptor |
| | Sortilin | Associates with p75 to mediate death signaling |
| Proliferation & | Notch 1-4 | Signaling receptor |
| Differentiation | Jagged 1, 2 | Notch ligand |
| | Delta 1, 2, 4 | Notch ligand |
| Growth | ErbB4 | Growth receptor |
| | GHR | Growth hormone receptor |

Here we describe experiments in which we examined the effect of inhibiting the GS complex on breast cancer cell lines using a peptide inhibitor (GSI1) and RNAi. Surprisingly and unexpectedly, we found that GSI1 was effective at killing breast cancer cell lines at a concentration of 1 μM but was only effective against normal breast cell lines at a concentration of over 50 μM. Furthermore, we found that inhibiting the production of the substrate binding component NCT was particularly effective in selectively targeting breast cancer cell lines. RNAi of GS components showed that only NCT RNAi caused inhibition of cell proliferation and consequent cell death in breast cancer cell lines, with minimal effect on normal breast cancer cells. In addition, NCT RNAi not only knocked out NCT but also the other components of GS at the molecular level with the NCT knockout also demonstrating upregulated Notch 2 expression, a known tumour suppressor gene. GS inhibitors, and in particular inhibitors of NCT, are therefore expected to be useful in combating breast cancer.

Again surprisingly and unexpectedly, we found a strong correlation between NCT and the metastatic potential of breast cancer tissue from the complete range of breast cancer patients in terms of hormone and Her-2 expression. NCT is therefore believed to be tightly correlated with metastatic potential and is likely to be a biomarker for breast cancer.

In neuronal cells there is evidence that the MAPK/ERK pathway can regulate the activity of GS by phosphorylation (Kim et al., 2006). In particular, it has been demonstrated in neural cells that inhibition of ERK1/2 increases GS activity by phosphorylation of NCT (Capell et al., 2005). Surprisingly, we found the same up-regulation of GS activity to occur in breast cancer cell lines. We postulate that this provides a mechanism by which breast cancer cells can become resistant to growth factor receptor inhibitors. Thus, targeting the GS complex via NCT is likely to restore sensitivity to growth factor receptor inhibitors and further contribute to the anti-proliferative effects of these drugs.

A first aspect of the invention provides a method of combating breast cancer in a mammalian individual, the method comprising administering to the individual an agent which reduces the function of the GS complex.

By “combating” breast cancer we include the meaning that the invention can be used to alleviate symptoms of the disorder (i.e. palliative use), or to treat the disorder, or to prevent the disorder (i.e. prophylactic use). We also include the meaning of reducing metastatic progression of breast cancer.

Preferably, the mammalian individual is a human. Alternatively, the individual may be an animal, for example a domesticated animal (for example a dog or cat), laboratory animal (for example laboratory rodent, mouse, rat or rabbit) or an animal important in agriculture (i.e. livestock), for example, cattle, sheep, horses or goats.

By GS complex we include the meaning of a complex formed from the products of a human NCT gene (mRNA sequence found in Genbank Accession No. NM_{—}015331), a human PS gene (mRNA sequence variants found in Genbank Accession Nos. NM_{—}000021, NM_{—}000447, NM_{—}012486), a human PEN-2 gene (mRNA sequence found in Genbank Accession No. NM_{—}172341), and a human APH-1 gene (mRNA sequence variants found in Genbank Accession Nos. NM_{—}001077628 and NM_{—}016022). It will be appreciated that there is natural variability with respect to the gene and mRNA sequences and this variability is included within the meaning of GS complex as herein defined.

By GS complex we also include an homologous complex from other mammalian species, comprising orthologues of human NCT, PS, PEN-2 and APH-1. Thus the invention includes the use of an agent which reduces the function of an homologous GS complex from other mammalian species. Examples of suitable homologous GS complexes include those from mice and rats. Mouse and rat NCT have 74.5% and 64.0% sequence identity with human NCT, respectively. Mouse and rat PS have 50.8% and 62.2% sequence identity with human PS, respectively. Mouse and rat PEN-2 have 59.5% and 61.8% sequence identity with human PEN-2, respectively. Mouse and rat APH-1 have 47.0% and 63.4% sequence identity with human APH-1, respectively. It will be appreciated that there is natural variability with respect to the gene and mRNA sequences encoding the orthologues of human NCT, PS, PEN-2 and APH-1, and this variability is included within the meaning of a homologous GS complex as herein defined.

With respect to the mammalian individual which is treated, it is preferred that the agent is one which reduces the function of the GS complex of that mammalian species. For example, when the mammalian individual is a human the agent reduces the function of human GS complex.

RNA interference (RNAi) experiments specific for GS components revealed that only nicastrin RNAi causes inhibition of cell proliferation and consequent cell death in breast cancer cell lines. Furthermore, nicastrin RNAi not only knocked out nicastrin but also the other components of GS at the molecular level.

Accordingly, in one embodiment of the invention, the agent which reduces the function of the GS complex is one which reduces the expression of nicastrin.

Various methods are available to reduce expression of specific genes including RNAi, antisense and triplet-forming oligoneucleotides, and ribozymes.

Thus, in a further embodiment the agent is a nicastrin siRNA molecule, a nicastrin antisense oligonucleotide or a nicastrin specific ribozyme.

RNAi is the process of sequence-specific post-transcriptional gene silencing in animals initiated by double stranded RNA (dsRNA) that is homologous in sequence to the silenced gene (siRNA; Hannon et al. Nature, 418 (6894): 244-51 (2002); Brummelkamp et al., Science 21, 21 (2002); and Sui et al., Proc. Natl Acad. Sci. USA 99, 5515-5520 (2002)). The mediators of sequence-specific mRNA degradation are typically 21- and 22-nucleotide small interfering RNAs (siRNAs) which, in vivo, may be generated by ribonuclease III cleavage from longer dsRNAs. 21-nucleotide siRNA duplexes have been shown to specifically suppress expression of both endogenous and heterologous genes (Elbashir et al (2001) Nature 411: 494-498). In mammalian cells it is believed that the siRNA has to be comprised of two complementary 21mers as described below since longer double-stranded RNAs (dsRNAs) will activate PKR (dsRNA-dependent protein kinase) and inhibit overall protein synthesis.

Duplex siRNA molecules selective for nicastrin can readily be designed by reference to its cDNA sequence (cDNA corresponding to human nicastrin mRNA found in Genbank Accession No. NM_{—}015331). Typically, the first 21-mer sequence that begins with an AA dinucleotide which is at least 120 nucleotides downstream from the initiator methionine codon is selected. The RNA sequence perfectly complementary to this becomes the first RNA oligonucleotide. The second RNA sequence should be perfectly complementary to the first 19 residues of the first, with an additional UU dinucleotide at its 3′ end. Once designed, the synthetic RNA molecules can be synthesised using methods well known in the art.

Examples of NCT RNAi molecules that may be used in the present invention include:

| | | | |
|---|---|---|---|
| 1. | Sense | | GAGAGCCGCUGGAAAGAUAUU |
| | AntiSense | 5′-P | UAUCUUUCCAGCGGCUCUCUU |
| 2. | Sense | | CCACUUAUGUUGUACAGUAUU |
| | AntiSense | 5′-P | UACUGUACAACAUAAGUGGUU |
| 3. | Sense | | GCACCAACCUUCCCACUAUUU |
| | AntiSense | 5′-P | AUAGUGGGAAGGUUGGUGCUU, |
| and | | | |
| 4. | Sense | | ACAAGGAUCUGUAUGAGUAUU |
| | AntiSense | 5′-P | UACUCAUACAGAUCCUUGUUU |

Antisense oligonucleotides are single-stranded nucleic acids, which can specifically bind to a complementary nucleic acid sequence. By binding to the appropriate target sequence, an RNA-RNA, a DNA-DNA, or RNA-DNA duplex is formed. The term “antisense” relates to the fact that they are complementary to the sense or coding strand of the gene. Recently, formation of a triple helix has proven possible where the oligonucleotide is bound to a DNA duplex. It was demonstrated that oligonucleotides could recognise sequences in the major groove of the DNA double helix to form a triple helix. This suggests that it is possible to synthesise a sequence-specific molecule which specifically binds double-stranded DNA via recognition of major groove hydrogen binding sites.

By binding to the target nucleic acid, antisense oligonucleotides can inhibit the function of the target nucleic acid. This may be a result of blocking the transcription, processing, poly(A)addition, replication, translation, or promoting inhibitory mechanisms of the cells, such as promoting RNA degradation.

Typically, antisense oligonucleotides are 15 to 35 bases in length. For example, 20-mer oligonucleotides have been shown to inhibit the expression of the epidermal growth factor receptor mRNA (Witters et al, Breast Cancer Res Treat 53:41-50 (1999)) and 25-mer oligonucleotides have been shown to decrease the expression of adrenocorticotropic hormone by greater than 90% (Frankel et al, J Neurosurg 91:261-7 (1999)). However, it is appreciated that it may be desirable to use oligonucleotides with lengths outside this range, for example 10, 11, 12, 13, or 14 bases, or 36, 37, 38, 39 or 40 bases. Antisense oligonucleotides specific for nicastrin can be designed by reference to the nicastrin cDNA sequence defined above using techniques well known in the art.

Ribozymes are RNA molecules capable of cleaving targeted RNA or DNA. Examples of ribozymes are described in, for example, Cech and Herschlag “Site-specific cleavage of single stranded DNA” U.S. Pat. No. 5,180,818; Altman et al “Cleavage of targeted RNA by RNAse P” U.S. Pat. No. 5,168,053; Cantin et al “Ribozyme cleavage of HIV-1 RNA” U.S. Pat. No. 5,149,796; Cech et al “RNA ribozyme restriction endoribonucleases and methods”, U.S. Pat. No. 5,116,742; Been et al “RNA ribozyme polymerases, dephosphorylases, restriction endonucleases and methods”, U.S. Pat. No. 5,093,246; and Been et al “RNA ribozyme polymerases, dephosphorylases, restriction endoribonucleases and methods; cleaves single-stranded RNA at specific site by transesterification”, U.S. Pat. No. 4,987,071, all incorporated herein by reference. Ribozymes specific for nicastrin can be designed by reference to the nicastrin cDNA sequence defined above using techniques well known in the art.

In addition to reducing the function of the GS complex by reducing the expression of nicastrin, the function of the GS complex can be reduced by inhibiting an enzymatic activity of the complex. Therefore, in another embodiment, the agent is one which inhibits an enzymatic activity of the GS complex.

Since the GS complex is an intramembrane aspartyl protease, it is preferred if the enzymatic activity of the complex inhibited by the agent is the aspartyl protease activity.

However, nicastrin also uses aminopeptidase activity to enable binding of the substrate. Thus it is also preferred if the enzymatic activity of the complex inhibited by the agent is the aminopeptidase activity.

In a particularly preferred embodiment, the agent is one which selectively inhibits an enzymatic activity of the GS complex.

By “selectively” inhibits we include the meaning that the agent has an IC₅₀ value for GS which is lower than for other proteins with the same enzymatic activity e.g. another aspartyl protease or another aminopeptidase.

Preferably, the agent has an IC₅₀ value at least five or ten times lower than for at least one other aspartyl protease (when considering the aspartyl protease activity) or at least one other aminopeptidase (when considering the aminopeptidase activity), and preferably more than 100 or 500 times lower. More preferably, the agent which selectively inhibits GS has an IC₅₀ value more than 1000 or 5000 times lower than for at least one other aspartyl protease (when considering the aspartyl protease activity) or at least one other aminopeptidase (when considering the aminopeptidase activity). Preferably, the at least one other aspartyl protease or at least one other aminopeptidase is a mammalian, more preferably human, aspartyl protease or aminopeptidase. Also preferably, the agent which selectively inhibits GS has a lower IC₅₀ value than for at least 2 or 3 or 4 or 5 or at least 10 other aspartyl proteases or aminopeptidases, as the case may be.

Most preferably, the agent which selectively inhibits GS has an IC₅₀ value at least five times lower than for all other human aspartyl proteases or aminopeptidases, and preferably at least 10, 50, 100 or 500 times lower.

Aspartyl proteases closely related to the GS aspartyl protease include signal peptide peptidase and presenilinase. Therefore, it is particularly preferred if the agent which selectively inhibits GS has an IC₅₀ value at least five times lower than for signal peptide peptidase or presenilinase, and preferably at least 10, 50, 100 or 500 times lower.

Various types of enzyme inhibitors exist with different modes of inhibition. For example, the agent may be a competitive inhibitor of GS, whereby the substrate and inhibitor compete for the same binding site or it may be a non-competitive inhibitor of GS, whereby the inhibitor binds to a different site on the enzyme molecule and reduces its enzymatic capacity. Inhibition may also be reversible or irreversible.

Thus in one embodiment the agent is selected from the group consisting of (i) an agent which binds to the GS complex substrate binding site; (ii) an agent which induces an inhibitory conformational change in the GS complex; (iii) an agent which modifies the active site; (iv) a neutralising antibody against the GS complex or a neutralising antibody against one of its components nicastrin, presenilin, presenilin enhancer-2 and anterior pharynx defective-1; and (v) an agent which sequesters the enzyme preventing its interaction with its substrate.

An example of a suitable agent which binds to the GS complex substrate binding site and therefore prevents substrate binding is GSI1 (Z-Leu-Leu-Nle-CHO), where Z indicates N-benzyloxycarbonyl. GSI1 is an amino acid analogue of the GS substrate which competitvely inhibits GS activity by binding to the active site of presenilin thus preventing binding of other substrates. Other peptide inhibitors of GS which inhibit GS in the same way include BOC-Gly-Val-Val-CHO and 2-Naphthoyl-Val-Phe-CHO.

An example of an agent that modifies the active site of the GS complex in such a way to prevent catalysis is DAPT (-N-[N-(3,5-Difluorophenacetyl-L-alanyl)]-S-phenylglycine t-butyl ester).

Further agents which inhibit GS include neutralising anti-GS antibodies or a neutralising antibody against one of its components nicastrin, presenilin, presenilin enhancer-2 and anterior pharynx defective i.e., those which inhibit the relevant enzymatic activity of GS. The term “antibody” includes but is not limited to polyclonal, monoclonal, chimaeric, single chain, Fab fragments and fragments produced by a Fab expression library. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. Furthermore, the antibodies and fragments thereof may be humanised antibodies, which are now well known in the art.

Other agents which inhibit GS include agents which although they do not inhibit the catalytic activity of GS directly, sequester GS and thereby prevent it binding to its substrate.

In a particularly preferred embodiment, the agent is the GS inhibitor GSI1 (Z-Leu-Leu-Nle-CHO).

Methods and assays for determining the activity of GS, and hence for determining whether and to what extent an agent inhibits GS, are described in Example 3.

The agent which reduces the function of the GS complex may be given to an individual who is being treated for breast cancer by some other means. Thus, although the method of treatment may be used alone, it is desirable to use it as an adjuvant therapy, for example alongside conventional preventative, therapeutic or palliative methods. Such methods may include surgery, radiation therapy including brachytherapy, and chemotherapy.

Thus, in an embodiment, the agent which reduces the function of the GS complex is administered to an individual who is also administered an additional anti-cancer agent. The individual may have been administered, the additional anti-cancer agent previously, or is administered the additional anti-cancer agent simultaneously with the agent which reduces the function of the GS complex, or is administered the additional anti-cancer agent after the agent which reduces the function of the GS complex.

Cancer chemotherapeutic agents include: alkylating agents including cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB); antibiotics such as dactinomycin (actinomycin D), daunorubicin (daunomycin; rubidomycin), doxorubicin, bleomycin, plicamycin (mithramycin) and mitomycin (mitomycin C); and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methyihydrazine, MIH); taxol, and analogues/derivatives; and hormone agonists/antagonists such as flutamide and tamoxifen.

We have demonstrated that treatment with an ERK inhibitor results in a 9 fold increase in GS activity in breast cancer cell lines which may give rise to resistance to ERK inhibitors. Therefore, reducing the function of the GS complex is likely to further contribute to the antiproliferative effects of drugs which target the MAPK/ERK signalling pathway.

Thus in a further embodiment, the additional anti-cancer agent is an inhibitor of the MAPK/ERK signalling pathway.

Examples of inhibitors of the MAPK/ERK signalling pathway include inhibitors of growth factor receptors, Ras, Raf, MEK1/2 and ERK1/2. Specific agents known to inhibit the MAPK/ERK signalling pathway include tyrosine kinase inhibitors, such as Gefitinib targeting EGFR and Lapatanib targeting HER2/EGFR and the ERK1/2 inhibitors U0126 (Madureira et al., 2006) and PD98059 (Lam et al., 2006).

In a particularly preferred embodiment the, inhibitor of the MAPK/ERK signalling pathway is the ERK1/2 inhibitor U0126.

Whilst it is possible for a therapeutic agent as described herein, to be administered alone, it is preferable to present it as a pharmaceutical formulation, together with one or more acceptable carriers. The carrier(s) must be “acceptable” in the sense of being compatible with the therapeutic molecule and not deleterious to the recipients thereof. Typically, the carriers will be water or saline which will be sterile and pyrogen free.

The formulations may conveniently be presented in unit dosage form and may be prepared by any of the methods well known in the art of pharmacy. Such methods include the step of bringing into association the active ingredient (for an antigenic molecule, construct or chimaeric polypeptide of the invention) with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Molded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured base, usually sucrose and acacia or tragacanth; pastilles comprising the active ingredient in an inert base such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and are preferably stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of the agent which reduces the function of the GS complex.

The amount of the agent which reduces the function of the GS complex which is administered to the individual is an amount effective to combat breast cancer. The amount may be determined by the physician.

We found that GSI1 was effective against breast cancer cell lines at a concentration of 1 μM but required concentrations in excess of 50 μM to be effective against normal breast cell lines. Therefore, typically, the dose of the agent which reduces the function of the GS complex to be administered is one that provides an effective concentration at the breast cancer of between 0.1 and 10 μM, preferably between 1 and 10 μM.

It should be understood that in addition to the ingredients particularly mentioned above, the formulations of this invention may include other agents conventional in the art having regard to the type of formulation in question, for example those suitable for oral administration may include flavouring agents.

In a preferred embodiment, the therapeutic agent is administered orally.

It will be appreciated that the therapeutic agent can be delivered to the area of the breast by any means appropriate for localised administration of a drug. For example, a solution of the therapeutic agent can be injected directly into the breast or can be delivered by infusion using an infusion pump. The therapeutic agent also can be incorporated into an implantable device which when placed at the desired site, permits the agent to be released into the surrounding locus.

The therapeutic agent may be administered via a hydrogel material. The hydrogel is non-inflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10% to about 80% by weight ethylene oxide and from about 20% to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename Pluronic®.

The agent which reduces the function of GS may also be targeted to the required site using a targeting moiety which binds to or lodges at the site of the breast cancer. A combined targeting/prodrug approach may also be useful. Moreover, a pro-drug approach may be used without targeting.

It is appreciated that the agent which reduces the function of the GS complex may itself be a polynucleotide, such as a nicastrin RNAi, a nicastrin antisense oligonucleotide or a nicastrin ribozyme. The agent may also be a polynucleotide encoding a polypeptide which reduces the function of the GS complex or a polynucleotide encoding a nicastrin RNAi, a nicastrin antisense oligonucleotide or a nicastrin ribozyme.

Polynucleotides may be administered by any effective method, for example, parenterally (eg intravenously, subcutaneously, intramuscularly) or by oral, nasal or other means which permit the oligonucleotides to access and circulate in the individual's bloodstream. Polynucleotides administered systemically, preferably are given in addition to locally administered polynucleotides, but also have utility in the absence of local administration. A dosage in the range of from about 0.1 to about 10 grams per administration to an adult human generally will be effective for this purpose.

The polynucleotide may be administered as a suitable genetic construct as is described below and delivered to the individual where it is expressed. Typically, the polynucleotide in the genetic construct is operatively linked to a promoter which can express the compound in the cell. The genetic constructs of the invention can be prepared using-methods well known in the art, for example in Sambrook et al (2001).

Although genetic constructs for delivery of polynucleotides can be DNA or RNA it is preferred if it is DNA.

Preferably, the genetic construct is adapted for delivery to a human cell.

Means and methods of introducing a genetic construct into a cell in an animal body are known in the art. For example, the constructs of the invention may be introduced into cells by any convenient method, for example methods involving retroviruses, so that the construct is inserted into the genome of the cell (Kuriyama et al (1991) Cell Struc. and Func. 16, 503-510). Alternatively, as described in Culver et al (1992) Science 256, 1550-1552, cells which produce retroviruses are injected. The retrovirus-producing cells so introduced are engineered to actively produce retroviral vector particles so that continuous productions of the vector occurred within the tumour mass in situ. Thus, proliferating epithelial cells can be successfully transduced in vivo if mixed with retroviral vector-producing cells.

Other methods involve simple delivery of the construct into the cell for expression therein either for a limited time or, following integration into the genome, for a longer time. An example of the latter approach includes liposomes (Nässander et al (1992) Cancer Res. 52, 646-653).

Other methods of delivery include adenoviruses carrying external DNA via an antibody-polylysine bridge (see Curiel (1993) Prog. Med. Virol. 40, 1-18) and transferrin-polycation conjugates as carriers (Wagner et al (1990) Proc. Natl. Acad. Sci. USA 87, 3410-3414).

The polynucleotide may also be delivered by adenovirus wherein it is present within the adenovirus particle, for example, as described below.

In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulfide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the tumour cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used.

It will be appreciated that “naked DNA” and DNA complexed with cationic and neutral lipids may also be useful in introducing the DNA of the invention into cells of the individual to be treated. Non-viral approaches to gene therapy are described in Ledley (1995) Human Gene Therapy 6, 1129-1144.

Alternative targeted delivery systems are also known such as the modified adenovirus system described in WO 94/10323 wherein, typically, the DNA is carried within the adenovirus, or adenovirus-like, particle. Michael et at (1995) Gene Therapy 2, 660-668 describes modification of adenovirus to add a cell-selective moiety into a fibre protein. Mutant adenoviruses which replicate selectively in p53-deficient human tumour cells, such as those described in Bischoff et al (1996) Science 274, 373-376 are also useful for delivering the genetic construct of the invention to a cell.

A second aspect of the invention provides the use of an agent which reduces the function of the GS complex in the manufacture of a medicament for combating breast cancer in a mammalian individual.

The invention includes the use of an agent which reduces the function of the GS complex and an additional anti-cancer agent, in the manufacture of a medicament for combating breast cancer in a mammalian individual.

The invention includes the use of an agent which reduces the function of the GS complex in the manufacture of a medicament for combating breast cancer in a mammalian individual, wherein the individual is administered an additional anti-cancer agent. Thus, the individual may have been administered the additional anti-cancer agent previously, or is administered the additional anti-cancer agent simultaneously with the medicament, or is administered the additional anti-cancer agent after the medicament.

The invention also includes the use of an anti-cancer agent in the manufacture of a medicament for combating breast cancer in a mammalian individual, wherein the individual is administered an agent which reduces the function of the GS complex. Thus, the individual may have been administered the agent which reduces the function of the GS complex previously, or is administered the agent which reduces the function of the GS complex simultaneously with the medicament, or is administered the agent which reduces the function of the GS complex after the medicament.

Preferences for the agent, the mammalian individual, the additional anti-cancer agent, routes of administration, formulations, and so on, in this and subsequent aspects of the invention are as described above with respect to the first aspect of the invention.

A third aspect of the invention provides an agent which reduces the function of the GS complex for use in combating breast cancer in a mammalian individual.

The invention includes an agent which reduces the function of the GS complex for use in combating breast cancer in a mammalian individual which agent further comprises an additional anti-cancer agent.

The invention includes an agent which reduces the function of the GS complex for use in combating breast cancer in a mammalian individual, wherein the individual is administered an additional anti-cancer agent. Thus, the individual may have been administered the additional anti-cancer agent previously, or is administered the additional anti-cancer agent simultaneously with the agent which reduces the function of the GS complex, or is administered the additional anti-cancer agent after the agent which reduces the function of the GS complex.

The invention also includes an anti-cancer agent for use in combating breast cancer in a mammalian individual, wherein the individual is administered an agent which reduces the function of the GS complex. Thus, the individual may have been administered the agent which reduces the function of the GS complex previously, or is administered the agent which reduces the function of the GS complex simultaneously with the anti-cancer agent, or is administered the agent which reduces the function of the GS complex after the anti-cancer agent.

A fourth aspect of the invention provides a method of inhibiting breast cancer cell proliferation in a mammalian individual, the method comprising administering to the individual an agent which reduces the function of the GS complex.

The individual may also be administered an additional anti-cancer agent. Thus, the individual may have been administered the additional anti-cancer agent previously, or is administered the additional anti-cancer agent simultaneously with the agent which reduces the function of the GS complex, or is administered the additional anti-cancer agent after the agent which reduces the function of the GS complex.

A fifth aspect of the invention provides the use of an agent which reduces the function of the GS complex in the manufacture of a medicament for inhibiting breast cancer cell proliferation.

The invention includes the use of an agent which reduces the function of the GS complex and an additional anti-cancer agent, in the preparation of a medicament for inhibiting breast cancer cell proliferation in a mammalian individual.

The invention includes the use of an agent which reduces the function of the GS complex in the manufacture of a medicament for inhibiting breast cancer cell proliferation in a mammalian individual, wherein the individual is administered an additional anti-cancer agent. Thus, the individual may have been administered the additional anti-cancer agent previously, or is administered the additional anti-cancer agent simultaneously with the medicament, or is administered the additional anti-cancer agent after the medicament.

The invention also includes the use of an anti-cancer agent in the manufacture of a medicament for inhibiting breast cancer cell proliferation in a mammalian individual, wherein the individual is administered an agent which reduces the function of the GS complex. Thus, the individual may have been administered the agent which reduces the function of the GS complex previously, or is administered the agent which reduces the function of the GS complex simultaneously with the medicament, or is administered the agent which reduces the function of the GS complex after the medicament.

A sixth aspect of the invention provides an agent which reduces the function of the GS complex for use in inhibiting breast cancer cell proliferation in a mammalian individual.

The invention includes an agent which reduces the function of the GS complex for use in inhibiting breast cancer cell proliferation in a mammalian individual which agent further comprises an additional anti-cancer agent.

The invention includes an agent which reduces the function of the GS complex for use in inhibiting breast cancer cell proliferation in a mammalian individual, wherein the individual is administered an additional anti-cancer agent. Thus, the individual may have been administered the additional anti-cancer agent previously, or is administered the anti-cancer agent simultaneously with the agent which reduces the function of the GS complex, or is administered the anti-cancer agent after the agent which reduces the function of the GS complex.

The invention also includes an anti-cancer agent for use in inhibiting breast cancer cell proliferation in a mammalian individual, wherein the individual is administered an agent which reduces the function of the GS complex. Thus, the individual may have been administered the agent which reduces the function of the GS complex previously, or is administered the agent which reduces the function of the GS complex simultaneously with the additional anti-cancer agent, or is administered the agent which reduces the function of the GS complex after the additional anti-cancer agent.

A seventh aspect of the invention provides a method of inhibiting breast cancer cell proliferation ex vivo, the method comprising contacting the cell with an agent which reduces the function of the GS complex.

The breast cancer cell may be an established breast cancer cell line, for example MCF-7, T47D, ZR-75-1, MDA-MB-231 or Cal-51, or may be a primary culture from a breast cancer biopsy.

In one embodiment, the method further comprises contacting the cell with an additional anti-cancer agent.

An eighth aspect of the invention provides a composition comprising an agent which reduces the function of the GS complex and an additional anti-cancer agent. The composition may be a pharmaceutical composition. Typically, the composition is for combating breast cancer.

Preferences for the agent which reduces the function of the GS complex and the additional anti-cancer agent are as described above with respect to the first aspect of the invention.

A ninth aspect of the invention is a kit of parts comprising an agent which reduces the function of the GS complex and an additional anti-cancer agent.

The kit of parts may also comprise instructions for use in a method of combating breast cancer or inhibiting breast cancer cell growth in a mammalian individual, or for use in a method of inhibiting breast cancer cell proliferation ex vivo.

Preferences for the agent which reduces the function of the GS complex and the additional anti-cancer agent are as described above with respect to the first aspect of the invention.

We have demonstrated that nicastrin expression is highly correlated with breast cancer metastatic potential when staining breast cancer tissue from the complete range of breast cancer patients in terms of hormone and Her-2 expression. Nicastrin is therefore considered to be associated with the development of breast cancer or the progression of breast cancer, for example, from a benign to a metastatic state. Accordingly, the amount and/or function of nicastrin and the amount of nucleic acid encoding nicastrin are likely to be key indicators for assessing an individual's risk of developing breast cancer, or for assessing the progression of breast cancer in an individual or for assisting in the diagnosis of breast cancer in an individual.

We have also shown that in benign breast cancer, nicastrin is expressed predominantly in the nucleus and not where it is active in the cytoplasm/plasma membrane as in metastatic breast cancer. Therefore, the cellular location of nicastrin is also likely to be a valuable marker for assessing an individual's risk of developing breast cancer, or for assessing the progression of breast cancer in an individual or for assisting in the diagnosis of breast cancer in an individual. Indeed, the inventors believe that determining the cellular location of nicastrin will provide information useful in deciding upon treatment options. If nicastrin is expressed at the plasma membrane the individual is likely to have metastatic breast cancer. If nicastrin is expressed in the nucleus, although this observation alone would not exclude the possibility of the individual having breast cancer, it would nevertheless aid in the diagnosis of breast cancer.

Thus, a tenth aspect of the invention is a method of assessing an individual's risk of developing breast cancer; or of assessing the progression of breast cancer in an individual; or for assisting in the diagnosis of breast cancer in a mammalian individual, comprising:
a) providing a biological sample from the individual, and
b) determining one or more of:
   i) amount and/or function of nicastrin
   ii) amount of nucleic acid encoding nicastrin
   iii) cellular location of nicastrin.

The invention includes the use of nicastrin in assessing an individual's risk of developing breast cancer.

The invention includes the use of nicastrin in assessing the progression of breast cancer in a mammalian individual.

The invention includes the use of nicastrin in assisting in the diagnosis of breast cancer in a mammalian individual.

Preferably, the mammalian individual is a human. Alternatively, the individual may be an animal, for example a domesticated animal (for example a dog or cat), laboratory animal (for example laboratory rodent, for example mouse, rat or rabbit) or an animal important in agriculture (i.e. livestock), for example cattle, sheep, horses or goats.

The biological sample from the individual may be any suitable sample. For example, the sample may be a fluid sample such as blood, serum, plasma or spinal fluid. It is appreciated that in order to determine the cellular location of nicastrin, the sample must be a tissue sample, for example breast tissue. The sample may be directly derived from the patient, for example, by biopsy of the tissue, or it may be derived from the patient from a site remote from the tissue, for example because cells from the tissue have migrated from the tissue to other parts of the body. Alternatively, the sample may be indirectly derived from the patient in the sense that, for example, the tissue or cells therefrom may be cultivated in vitro, or cultivated in a xenograft model; or the nucleic acid sample may be one which has been replicated (whether in vitro or in vivo) from nucleic acid from the original source from the patient. Thus, although the nucleic acid derived from the patient may have been physically within the patient, it may alternatively have been copied from nucleic acid which was physically within the patient. The tumour tissue may be taken from the primary tumour or from metastases.

It is appreciated that for many of these methods there may be the need for a “reference sample”, i.e. a biological sample taken from an individual who does not have breast cancer.

A range of methods are available to determine the amount of nicastrin in a biological sample.

Preferred for assaying nicastrin polypeptide levels in a biological sample are antibody-based techniques. For example, nicastrin polypeptide expression can be studied with classical immunohistological methods. In these, the specific recognition is provided by the primary antibody (polyclonal or monoclonal) but the secondary detection system can utilise fluorescent, enzyme, or other conjugated secondary antibodies. As a result, an immunohistological staining of a tissue section for pathological examination is obtained (see, for example, Example 2). Tissues can also be extracted, e.g., with urea and neutral detergent, for the liberation of nicastrin polypeptide for Western-blot or dot/slot assay (Jalkanen, M., et al., J. Cell. Biol. 101:976-985 (1985); Jalkanen, M., et al., J. Cell. Biol. 105:3087-3096 (1987)). In this technique, which is based on the use of cationic solid phases, quantification of nicastrin can be accomplished using isolated nicastrin as a standard. The technique can also be applied to fluid samples. With these samples, a molar concentration of nicastrin will aid to set standard values of nicastrin content for different mammalian fluids. The normal range of nicastrin amounts can then be defined using values from healthy individuals, which can be compared to those obtained from a test subject.

Other antibody-based methods useful for detecting nicastrin polypeptide gene expression include immunoassays, such as the enzyme linked immunosorbent assay (ELISA) and the radioimmunoassay (RIA). For example, a nicastrin-specific monoclonal antibody can be used both as an immunoadsorbent and as an enzyme-labelled probe to detect and quantify nicastrin. The amount of nicastrin present in the sample can be calculated by reference to the amount present in a standard preparation using a linear regression computer algorithm. Such an ELISA for detecting a tumour antigen is described in lacobelli et al., Breast Cancer Research and Treatment 11: 19-30 (1988). In another ELISA assay, two distinct specific monoclonal antibodies can be used to detect nicastrin. In this assay, one of the antibodies is used as the immunoadsorbent and the other as the enzyme-labelled probe.

In addition to assaying nicastrin polypeptide levels in a biological sample, nicastrin can also be detected in vivo by imaging. Antibody labels or markers for in vivo imaging of nicastrin include those detectable by X-radiography, NMR, ESR or PET (Positron Emission Tomography). For X-radiography, suitable labels include radioisotopes such as barium or caesium, which emit detectable radiation but are not overtly harmful to the subject. Suitable markers for NMR and ESR include those with a detectable characteristic spin, such as deuterium, which may be incorporated into the antibody by labelling of nutrients for the relevant hybridoma. Suitable markers for PET include those labelled with a short lived radioactive tracer isotope such as ¹¹C or ¹⁸F.

Nicastrin-specific antibodies for use in the screening methods of the present invention can be raised against the intact nicastrin or an antigenic polypeptide fragment thereof, which may be presented together with a carrier protein, such as an albumin, to an animal system (such as rabbit or mouse) or, if it is long enough (at least about 25 amino acids), without a carrier.

Commercially available anti-nicastrin antibodies can be used in the present invention. For example, the anti-nicastrin antibody raised against an immunogenic peptide from the C-terminus of the protein is available from Sigma-Aldrich.

The step of determining the function of nicastrin may be performed using various techniques known to those skilled in the art. For example, the substrate binding activity of the GS complex containing nicastrin (for example immunoprecipitated from a patient sample using antibodies directed to nicastrin) may be assessed. Nicastrin substrates could be identified by an in vitro pull-down assay, a well known technique to those skilled in the art, to determine the physical interaction between the GS complex and its substrate. Moreover, the activity of the GS complex could be assessed as described, for example, in Example 3.

The step of determining the amount of nucleic acid encoding nicastrin may be performed using a number of different methods.

Levels of mRNA encoding nicastrin may be assayed using the RT-PCR method described in Makino et al., Technique 2:295-301 (1990). In this method, the specific mRNA is reverse transcribed into DNA which is then amplified such that the final DNA concentration is proportional to the initial concentration of target mRNA. Variations on this RT-PCR method will be apparent to the person skilled in the art. Any set of oligonucleotide primers which will amplify reverse transcribed target mRNA can be used and can be designed as will be well known to those skilled in the art. Alternative techniques by which to measure mRNA levels include incorporation of SybrGreen or other fluorophores into primers or probes as part of RealTime PCR experiments.

Another method used to measure levels of mRNA encoding nicastrin is northern blotting, a method well known to those skilled in the art and described further in Sambrook et al., 2001.

Further methods which may be of use in measuring mRNA levels include in situ hybridisation (In Situ Hybridization Protocols. Methods in Molecular Biology Volume 33. Edited by K H A Choo. 1994, Humana Press Inc (Totowa, N.J., USA) pp 480p and In Situ Hybridization: A Practical Approach. Edited by D G Wilkinson. 1992, Oxford University Press, Oxford, pp 163), in situ amplification, nuclease protection, probe arrays, and amplification based systems.

Various methods are available to determine the cellular location of nicastrin, for example using immunohistological analyses, for example, as described above and which are well known in the art.

If the sample has an increased amount and/or function of nicastrin, or if the sample has an increased amount of nucleic acid encoding nicastrin or if nicastrin in the sample is expressed at the plasma membrane, then the individual is considered to be at risk of developing breast cancer; or the individual is considered to have breast cancer that is progressing to a metastatic state; or the individual is considered to have breast cancer.

It will be appreciated that determination of the level of nicastrin in the sample will be useful to the clinician in determining how to manage the cancer in the patient. For example, since we have demonstrated that elevated levels of nicastrin are associated with the metastatic potential of breast cancer, the clinician may use the information concerning the levels of nicastrin to facilitate decision making regarding treatment of the patient. Thus, if the level of nicastrin is indicative of a low metastatic potential of the breast cancer, unnecessary radical surgery may be avoided. Similarly, if the level of nicastrin is indicative of a high metastatic potential of breast cancer radical surgery (i.e. mastectomy) may be the preferred treatment. Even if it is not appropriate to alter the type of surgery carried out, determining whether the level of nicastrin is indicative of a high metastatic potential may help the clinician decide whether the patient needs adjuvant systemic treatment or not. At present, a major aim in oncology is to be able to distinguish those breast cancers with a high metastatic potential from those with a low metastatic potential, because those with a low metastatic potential should not need to be put through six months of very toxic chemotherapy treatment.

The amount of nicastrin, function of nicastrin, and the amount of nucleic acid encoding nicastrin that is indicative of a risk of an individual developing breast cancer may vary depending on the type of individual (e.g. human, horse, dog and so on). The amount or function may be determined in each case by comparing the amount/function in individuals which subsequently went on to develop breast cancer with the amount/function in normal individuals (i.e. those with no signs or symptoms of breast cancer). Typically, the amount/function which is indicative of a risk of developing breast cancer is one which is greater than 2 standard deviations above the mean for the normal range (i.e. in individuals with no signs or symptoms of breast cancer).

The amount of nicastrin, function of nicastrin, and the amount of nucleic acid encoding nicastrin that is indicative of an individual having breast cancer that is progressing to a metastatic state may also vary depending on the type of individual (e.g. human, horse, dog and so on). The amount or function may be determined in each case by comparing the amount/function in individuals with benign breast cancer with the amount/function in individuals with metastatic breast cancer. Typically, the amount/function which is indicative of an individual having breast cancer that is progressing to a metastatic state is one which is greater than 2 standard deviations above the mean for individuals with benign breast cancer.

The amount of nicastrin, function of nicastrin, and the amount of nucleic acid encoding nicastrin that is indicative of an individual which has breast cancer may vary depending on the type of individual (e.g. human, horse, dog and so on). The amount or function may be determined in each case by comparing the amount/function in the individual with the amount/function in normal individuals (i.e. those with no signs or symptoms of breast cancer). Typically, the amount/function which is indicative of an individual having breast cancer is one which is greater than 2 standard deviations above the mean for the normal range (i.e. in individuals with no signs or symptoms of breast cancer).

An eleventh aspect of the invention provides the use of an agent which is capable of being used in determining one or more of: (i) the amount and/or function of nicastrin; (ii) the amount of nucleic acid encoding nicastrin; and (iii) the cellular location of nicastrin, in the manufacture of a reagent for assessing an individual's risk of developing breast cancer; or for assessing the progression of breast cancer in an individual; or for assisting in the diagnosis of breast cancer in an individual.

Examples of agents that can be used in this aspect of the invention to determine the amount and/or function of nicastrin include antibodies or compounds that can bind to nicastrin. Furthermore, as described in the tenth aspect of the invention, agents that may be used to assess the function of nicastrin include agents useful in measuring GS substrate binding and activity.

Agents that can be used in this aspect of the invention to determine the amount of nucleic acid encoding nicastrin include nucleic acid molecules which hybridise preferably selectively to the nucleic acid encoding nicastrin, such as primers, oligonucleotides, or other nucleic acid molecules useful in PCR-based methods, northern blotting and in situ hybridisation methods as described in the tenth aspect of the invention.

Agents that can be used in this aspect of the invention to determine the cellular location of nicastrin include antibodies or compounds that can bind to nicastrin.

A twelfth aspect of the invention provides an agent which is capable of being used in determining one or more of: (i) the amount and/or function of nicastrin; (ii) the amount of nucleic acid encoding nicastrin; and (iii) the cellular location of nicastrin, for use in assessing an individual's risk of developing breast cancer; or for assessing the progression of breast cancer in an individual; or for assisting in the diagnosis of breast cancer in an individual.

Suitable agents included in this aspect of the invention are as described above in the eleventh aspect of the invention.

A thirteenth aspect of the invention is a method of identifying a compound which may be useful in the treatment of breast cancer, comprising determining whether a test compound modulates at least one of: the amount and/or function of nicastrin, the amount of nucleic acid encoding nicastrin or the cellular location of nicastrin.

Suitable methods of determining the amount and/or function of nicastrin, the amount of nucleic acid encoding nicastrin and the cellular location of nicastrin are as described above with respect to the eleventh aspect of the invention.

The invention includes screening methods to identify drugs or lead compounds of use in the treatment of breast cancer. It is appreciated that screening assays which are capable of high throughput operation are particularly preferred.

It is appreciated that in the methods described herein, which may be drug screening methods, a term well known to those skilled in the art, the test compound may be a drug-like compound or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons and which may be water-soluble. A drug-like compound may additionally exhibit features of selective interaction with a particular protein or proteins and be bioavailable and/or able to penetrate target cellular membranes or the blood:brain barrier, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics. Suitable lead compounds for use in the current invention include aminopeptidase inhibitors.

Accordingly, in one embodiment the method further comprises: selecting a test compound which modulates at least one of (i) the amount and/or function of nicastrin, (ii) the amount of nucleic acid encoding nicastrin and (iii) the cellular location of nicastrin; modifying the test compound; and testing the ability of the modified compound to modulate at least one of (i) the amount and/or function of nicastrin, (ii) the amount of nucleic acid encoding nicastrin and (iii) the cellular location of nicastrin.

Once a candidate compound has been identified which modulates at least one of (i) the amount and/or function of nicastrin, (ii) the amount of nucleic acid encoding nicastrin and (iii) the cellular location of nicastrin, it may be desirable to test its effect in a suitable cell line in vitro. Examples of suitable breast cancer cell lines include MCF-7, T47D, ZR-75-1, MDA-MB-231 and Cal-51.

Thus in one embodiment, the method is performed in vitro. By in vitro we include both cell-free and cell-based assays.

It is also appreciated that it may be desirable to test the effect of a candidate compound in an animal model of breast cancer. Suitable experimental models of breast cancer include inducible transgenic mouse models (Chodosh et al., 2003) and the rat NMU model (Russo and Russo, 1996).

Accordingly, in another embodiment, the method is performed in vivo.

All of the documents referred to herein are incorporated herein, in their entirety, by reference.

The invention will now be described in more detail with the aid of the following Figures and Examples.

FIG. 1: Topology of Presenilin

FIG. 2: Topology of Anterior Pharnyx Defective (APH1)

FIG. 3: Topology of Presenilin Enhancer Protein-2 (PEN-2)

FIG. 4: Nicastrin topology and domain structure

(A) Topology of Nicastrin (B) Domain structure of Nicastrin illustrating position of signal peptide (SP), DAP domain and transmembrane (TM) domain.

FIG. 5: Proposed mechanism of GS action

Proposed role of nicastrin in GS complex mediated regulated intramembrane proteolysis (taken from Shah et al., 2005). GS substrates typically consist of type I membrane proteins with small extracellular stubs resulting from ectodomain shedding of the precursor proteins. Nicastrin is believed to act via its large ectodomain as a receptor for GS substrates by directly interacting with the free N termini of the substrates. The extracellular DAP domain of nicastrin, in which the conserved Glu333 residue and the DYIGS motif are critical, is involved in substrate recognition.

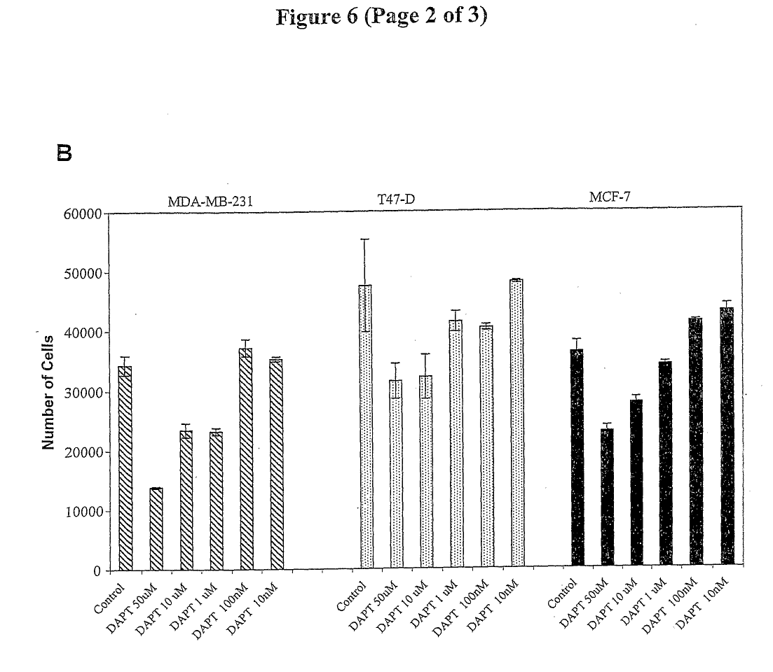

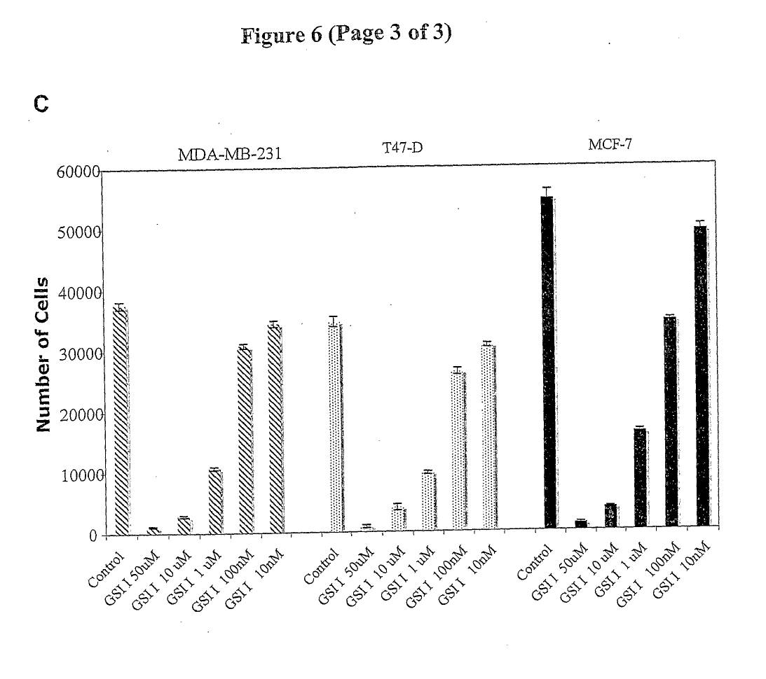

FIG. 6: Effect of three GS inhibitors (GSIs) on breast cancer cell line proliferation

Graphs illustrating the effects of three GSIs: (A) DAPT (N-[N-(3,5-Difluorophenacetyl-L-alanyl)]-S-phenylglycine t-butyl ester), (B) Compound E ((2S)-2-{[(3,5-Difluorophenyl)acetyl]amino}-N-[(3S)-1-methyl-2-oxo-5-phenyl-2,3-dihydro-1H-1,4-benzodiazepin-3-yl]propanamide) and (C) GSI1 (Z-Leu-Leu-Nle-CHO) on proliferation in three breast cancer cell lines (MDA-MB-231, T47-D and MCF-7 cells). Cells were treated with the GSIs at different concentrations for 72 hours and the number of cells counted.

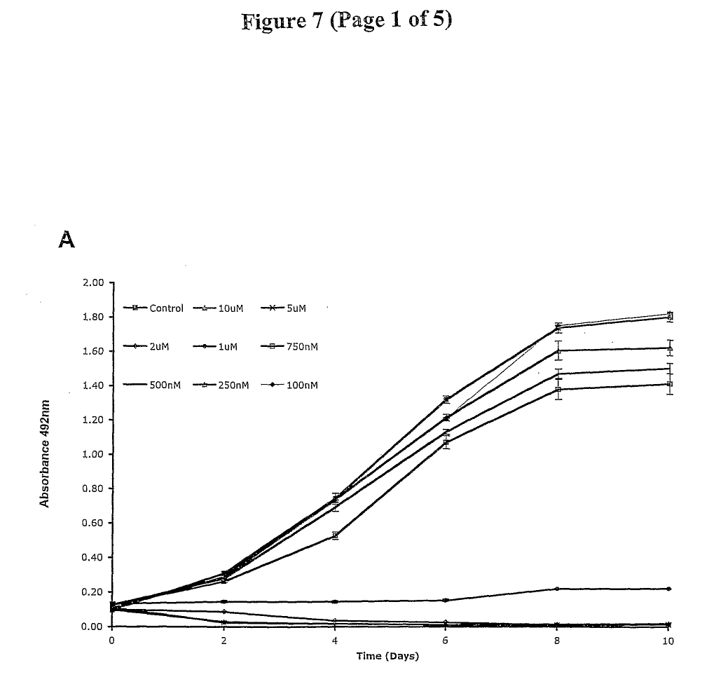

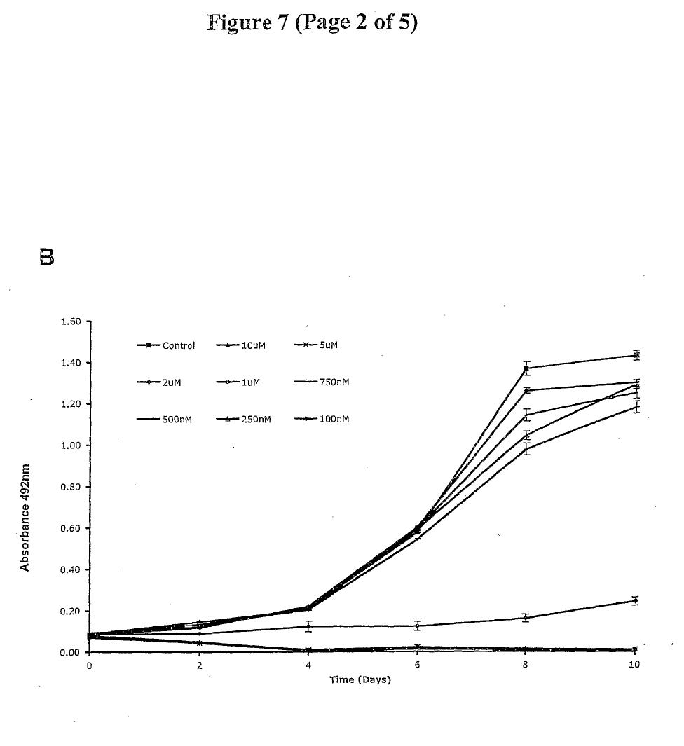

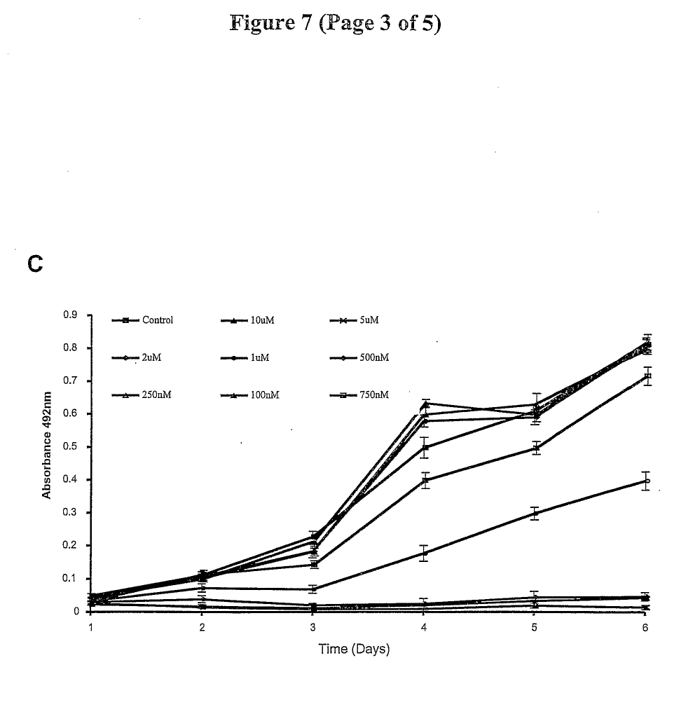

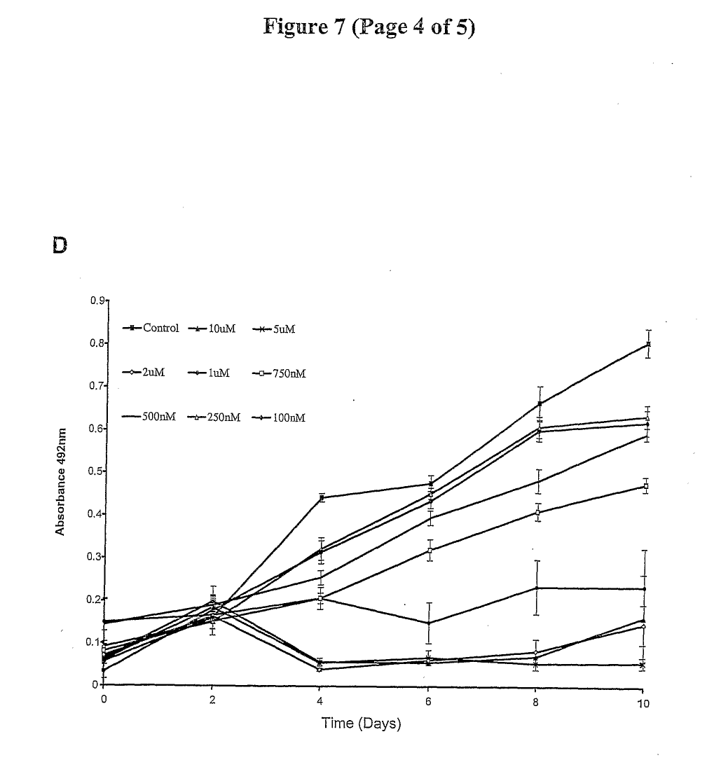

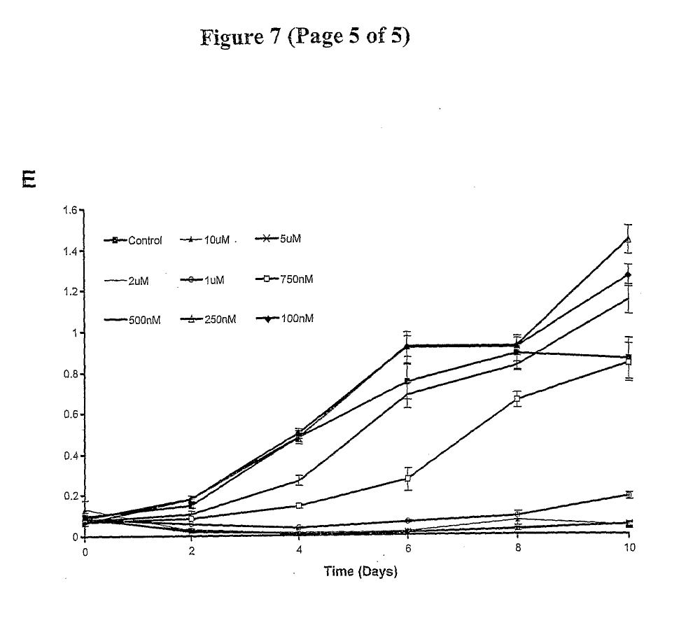

FIG. 7: Effect of GSI1 on breast cancer cell lines

Graphs illustrating the effects of GSI1, at different concentrations, on proliferation in five breast cancer cell lines: (A) MCF-7 cells, (B) T47D cells, (C) ZR-75-1 cells, (D) MDA-MB-231 cells and (E) Cal- 51 cells.

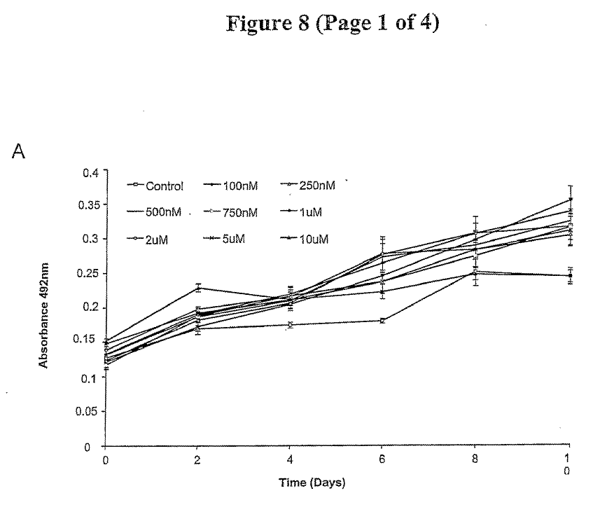

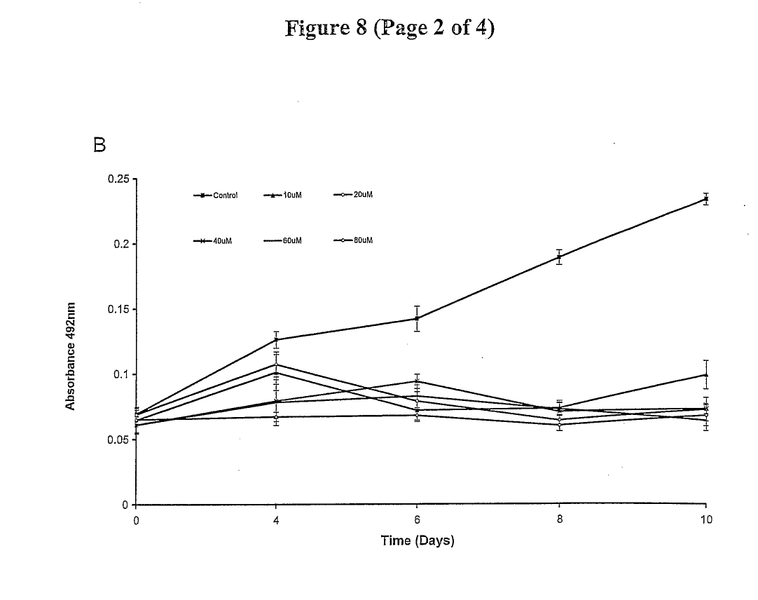

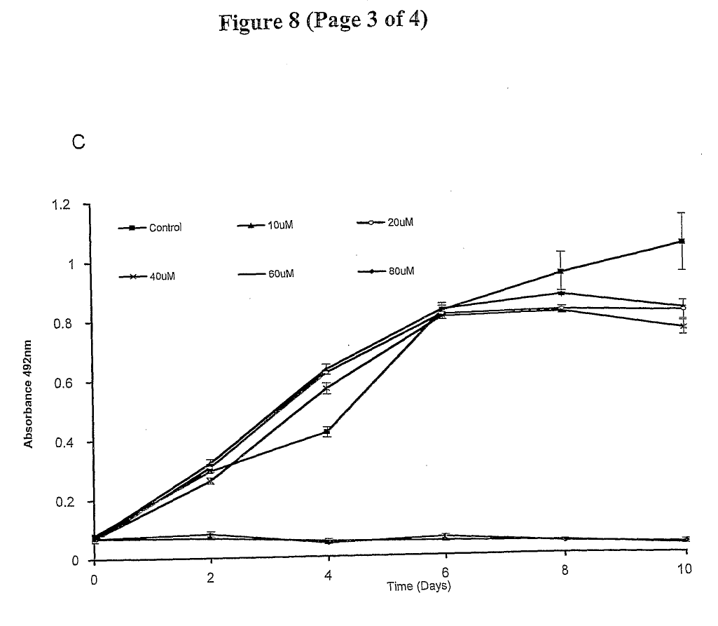

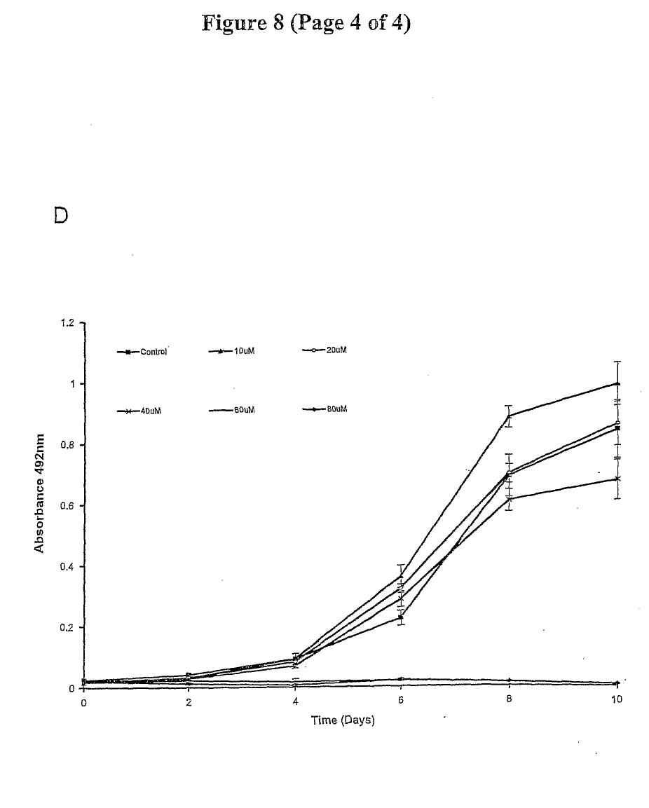

FIG. 8: Effect of GSI1 on normal cell lines

Graphs illustrating the effects of GSI1, at different concentrations, on proliferation in four normal cell lines: (A) MCF-10A cells, (B) MCF-10A, (C) L226U19 cells and (D) L226TS4 cells.

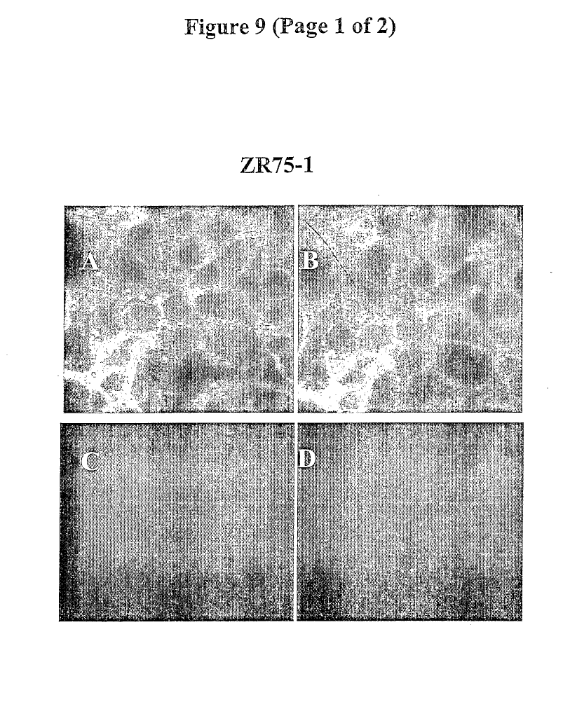

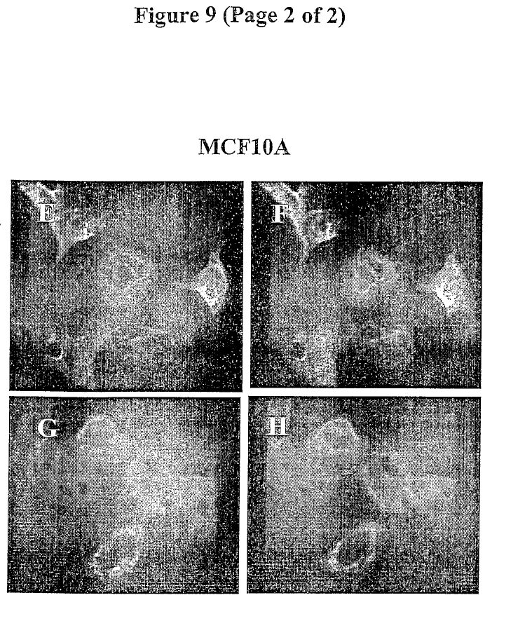

FIG. 9: GSI1 increases concentration of the cell adhesion molecule CD44

Stained sections of the breast cancer cell line ZR75-1 (A-D) and the normal cell line (E-H) showing effect of GSI1 on CD44 concentration. Upper sections (A, B, E and F) show cells treated with GSI1. Lower sections (C, D, G and H) show untreated cells. Sections were stained with an anti CD44 primary antibody and a fluorescently labeled secondary antibody to detect CD44. Nuclei were counterstained with DAPI, as shown in panels A, C, E and G.

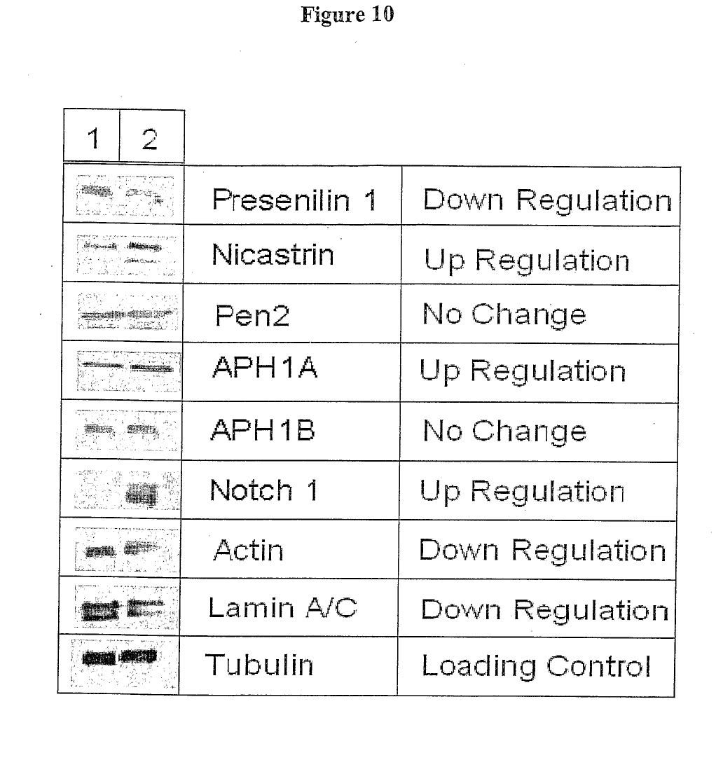

FIG. 10: GSI has little effect of protein levels apart from Notch 1

Western blot analysis of GS components, Notch 1, Actin and Lamin A/C in response to DMSO control (1) or 750 nm GSI1 (2). Tubulin was used as a loading control. GS inhibition caused only marginal effects on protein expression apart from Notch 1 expression which was markedly upregulated.

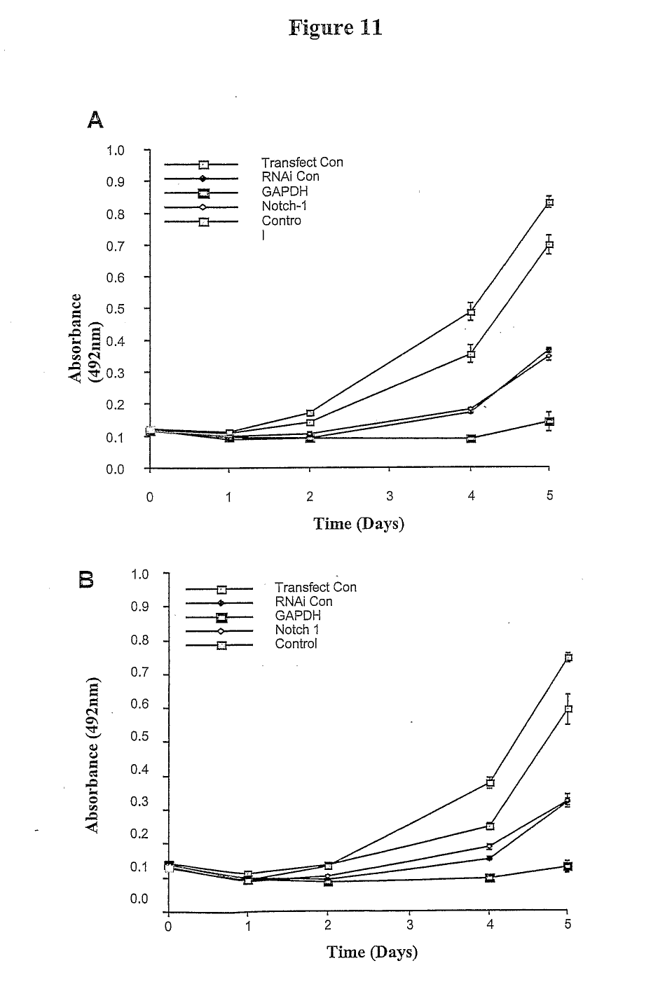

FIG. 11: Effect of RNAi Notch 1 on breast cancer cell lines

Graphs illustrating effect of RNAi Notch 1 on cell proliferation in (A) MCF-7 cell lines and (B) MDA-MB-231 cell lines. Cells were treated with the transfection reagent oligofectamine (transfection control), a non specific RNAi from a bacterial sequence (RNAi control), an RNAi sequence for Glyceraldehyde-3-phosphate dehydrogenase (GAPDH as positive control) and an RNAi specific for Notch 1. Untreated cells acted as the control for normal cell proliferation.

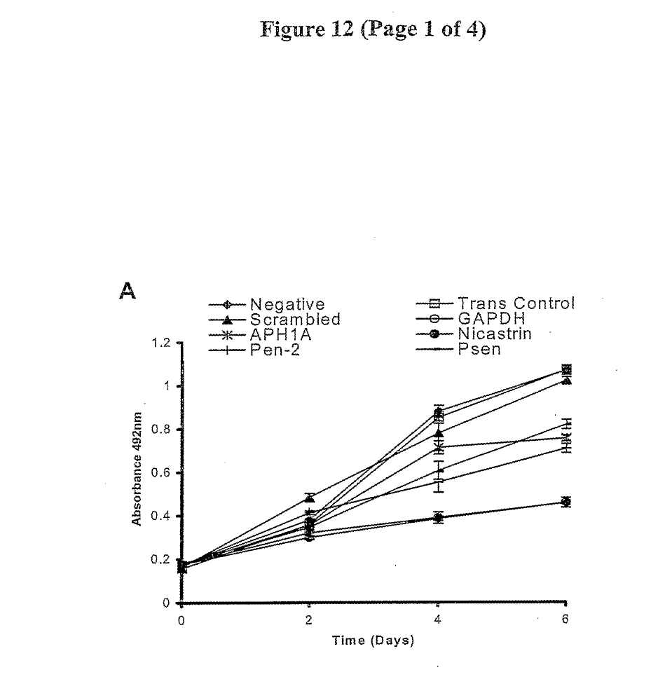

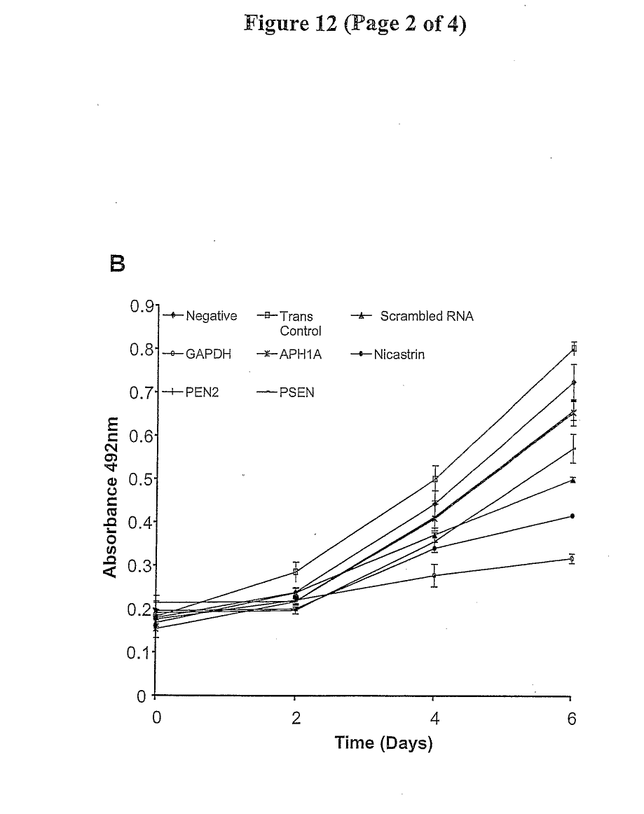

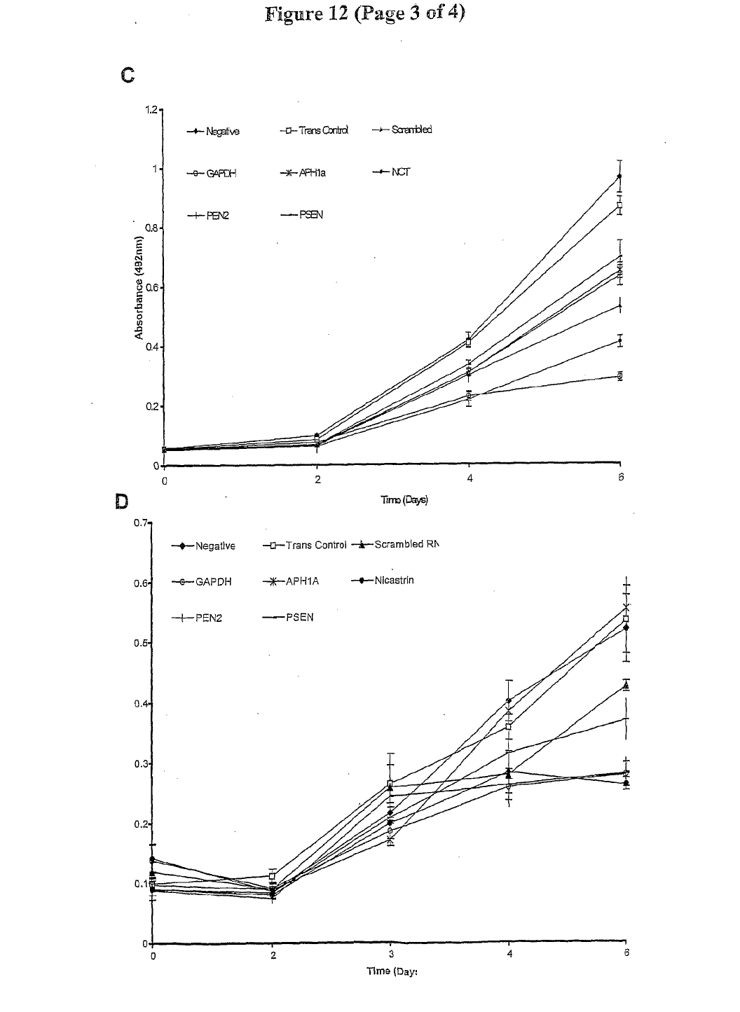

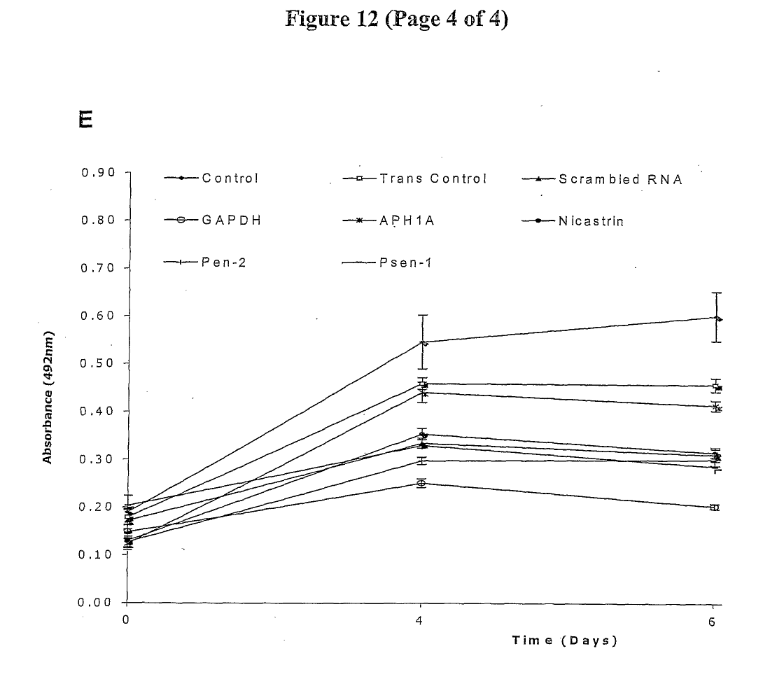

FIG. 12: RNAi for GS components in breast cancer and normal cell lines

Graphs illustrating effects of RNAi specific for APH-1A, Presenilin1, PEN-2 and nicastrin on cell proliferation in (A) MCF7, (B) MDA-MB-231, (C) ZR-71-1 and (D) T47D, breast cancer cell lines and in (E) MCF10A normal cell lines. Untreated cells acted as the control for normal cell proliferation (negative).

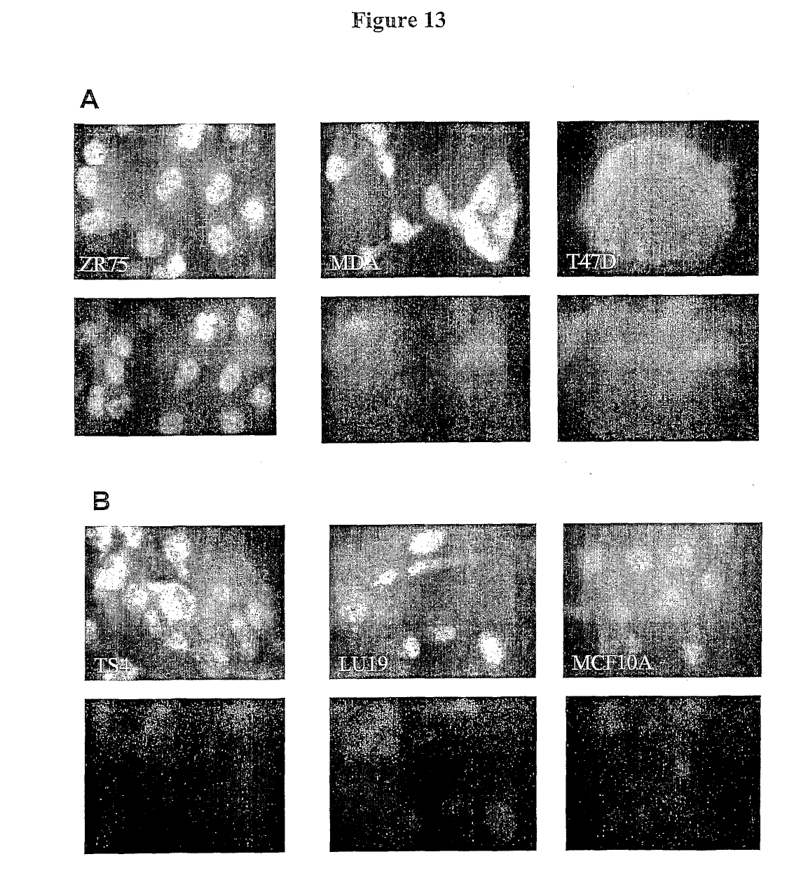

FIG. 13: NCT expression in normal and breast cancer cell lines

Sections of breast cell lines (A) and normal cell lines (B) stained for nicastrin expression (upper panels in A and B). Lower panels indicate nuclei as shown by the blue DAPI stain.

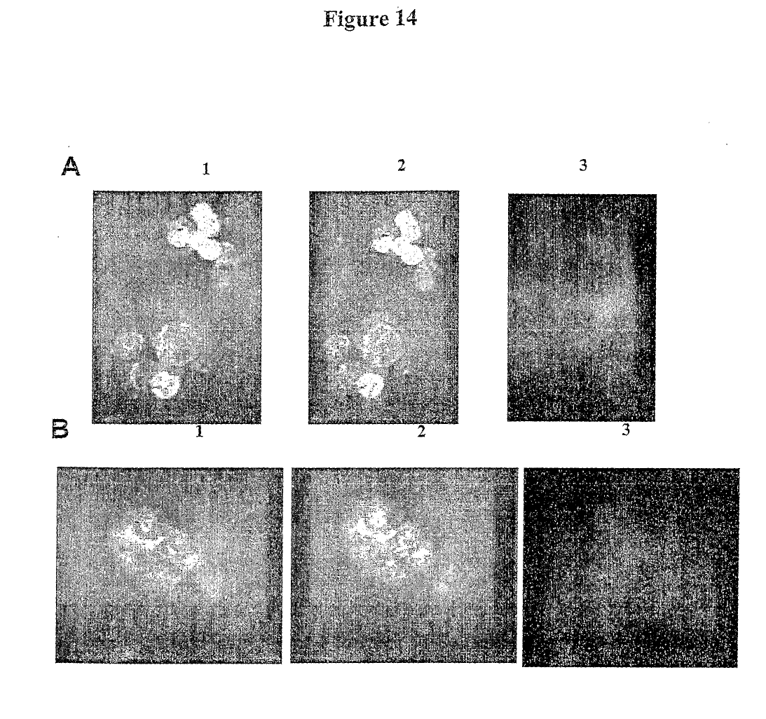

FIG. 14: NCT expression in “benign” breast cancer cells and breast cancer stem cells

Sections of (A) breast cancer stem cells, defined by those cells which express epithelial cell adhesion molecule (Ep-CAM) positive, CD44 positive and CD24 low/negative Ep-CAM⁺/CD44⁺/CD24^{low/-} and (B) Ep-CAM⁺/CD44⁺/CD24⁺ cells which can be defined as benign breast cancer or non-stem cell breast cancer cells, stained for expression of nicastrin. Panel 1 illustrates nicastrin staining, panel 2 illustrates nicastrin and nuclear staining and panel 3 illustrates nuclear staining, as shown by the blue DAPI stain.

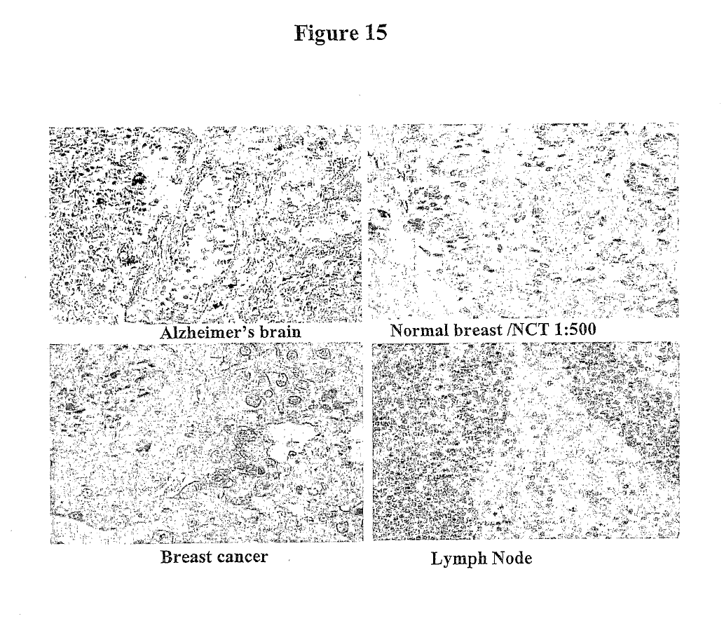

FIG. 15: NCT expression in Alzheimer's brain tissue, normal breast tissue, breast cancer tissue and a lymph node

Sections of tissues from an Alzheimer's brain, normal breast, breast cancer tissue and lymph node, stained for nicastrin expression.

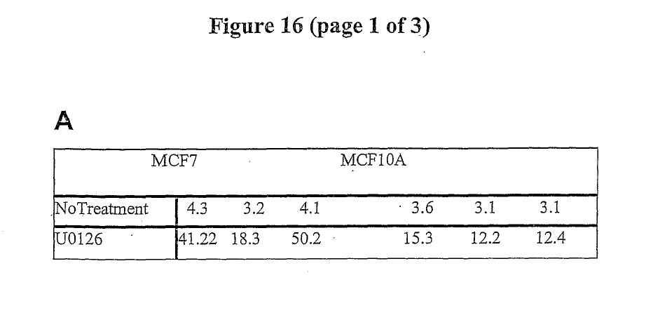

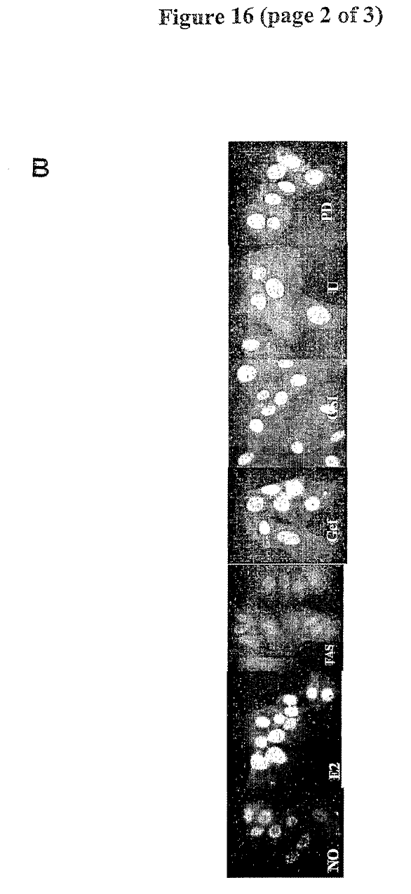

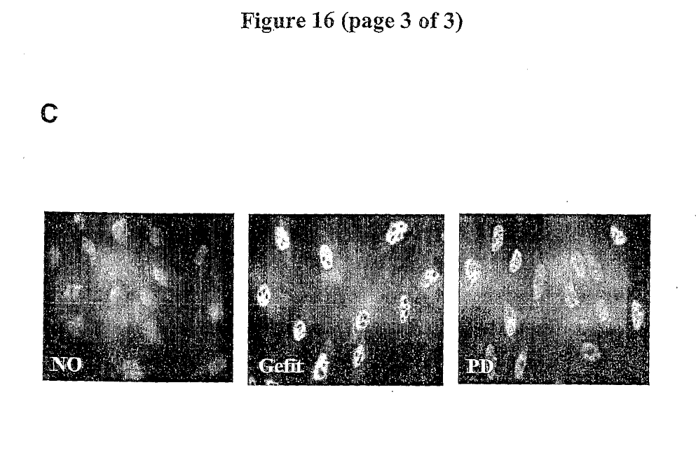

FIG. 16: The effect of the MAPK/ERK inhibitor 00126 on GS activity in normal and breast cancer cell lines.

(A) GS activity as measured by a fluorescent assay in MCF7 breast cancer cell lines and MCF10A normal cell lines, with and without treatment with the ERK1/2 inhibitor U0126. (B) Sections of MCF7 breast cancer cell lines stained for notch intracellular domain expression (as a surrogate marker for GS activity). Shown are sections of cells untreated (NO), and cells treated with oestrogen (E2), faslodex (FAS), Gefitinib (Gef), GSI1, and the ERK1/2 inhibitors U0126 (U) and. PD98059 (PD). (C) Sections of MCF10A normal cell lines stained for notch intracellular domain expression. Shown are sections of cells untreated (NO), and cells treated with Gefitinib (Gef) and PD98059 (PD).

FIG. 17: Nicastrin staining in breast tissue

A) Breast cancer cases showing gradation of NCT expression×200, i=+, ii=++, iii=+++; B) Normal breast tissue×200; C) Fibroadenoma×200; D) is TMA, ii TMA+competing peptide×100; E) i: breast carcinoma and ii: corresponding lymph node×200; F) i: Breast carcinoma and ii: adjacent normal breast tissue×200.

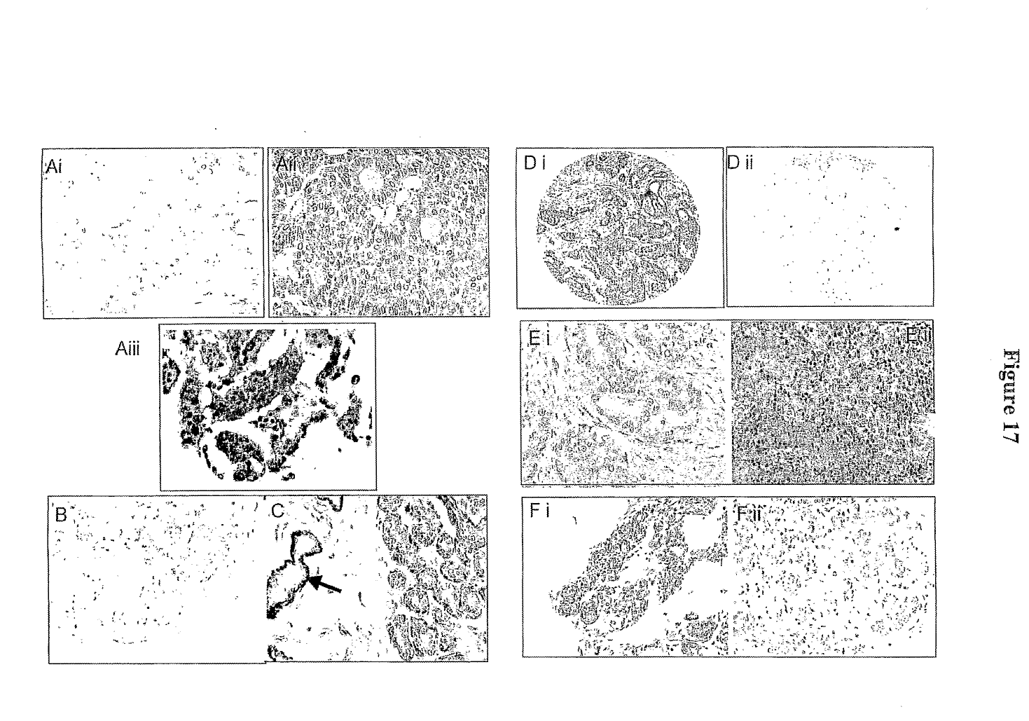

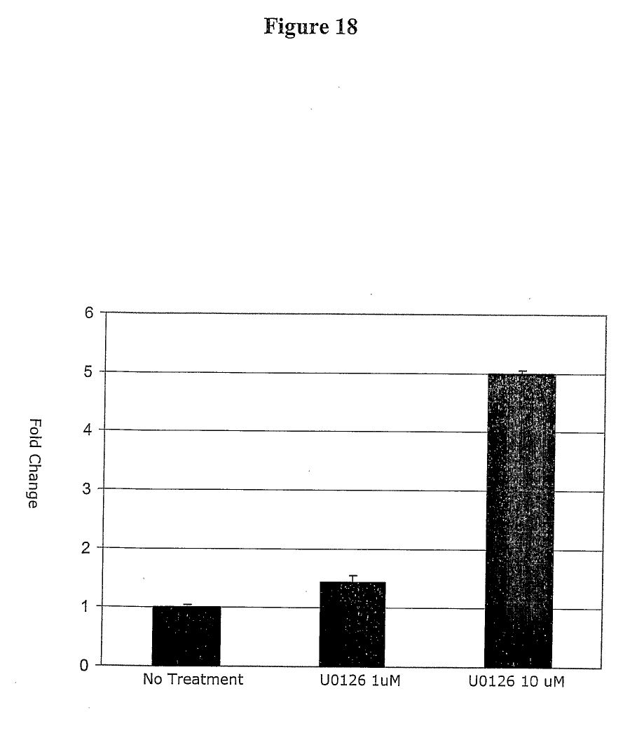

FIG. 18: Gamma-Secretase Activity Assay and CLS Reporter Assay upon ERK1/2 inhibitor (U0126) treatment:

GS Activity Assay was performed on MDA-MB 231 cell line. Cells were treated with a vehicle control and a U0126 inhibitor for 48 h. Results indicated that inhibition of ERK1/2 by U0126 in MDA-MB 231 cell line causes a dose dependant increase of GS Activity, strengthening the hypothesis of an existing cross-talk between the GS and the MAPK/ERK signalling pathways.

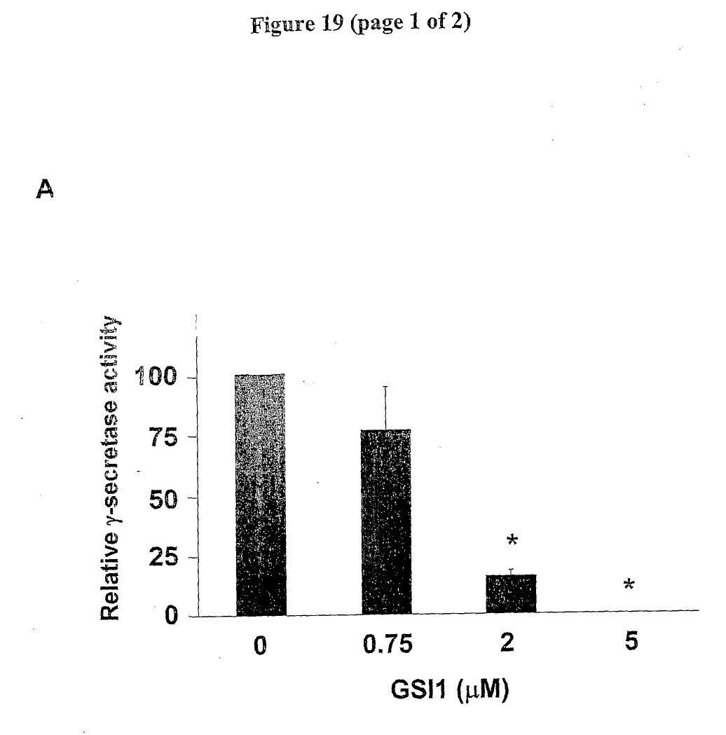

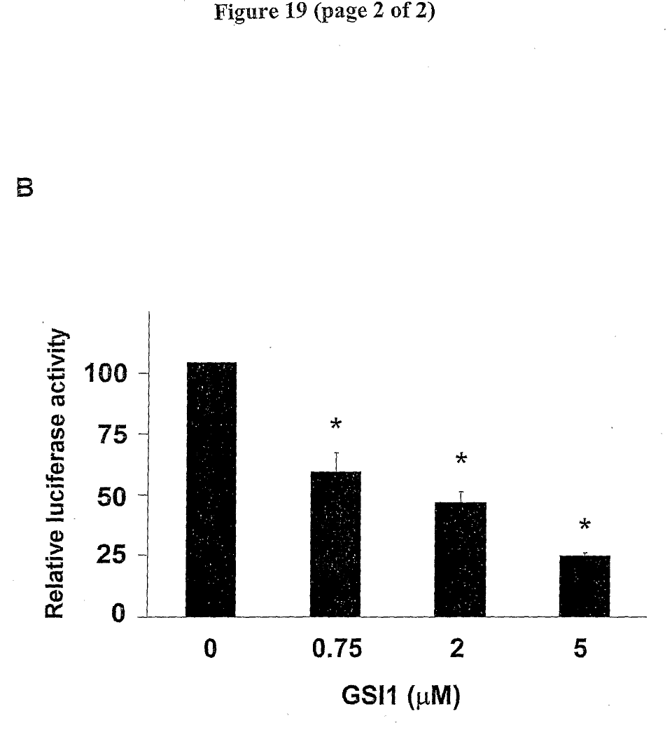

FIG. 19: Gamma-Secretase Activity Assay and CLS Reporter Assay upon GSI1 treatment:

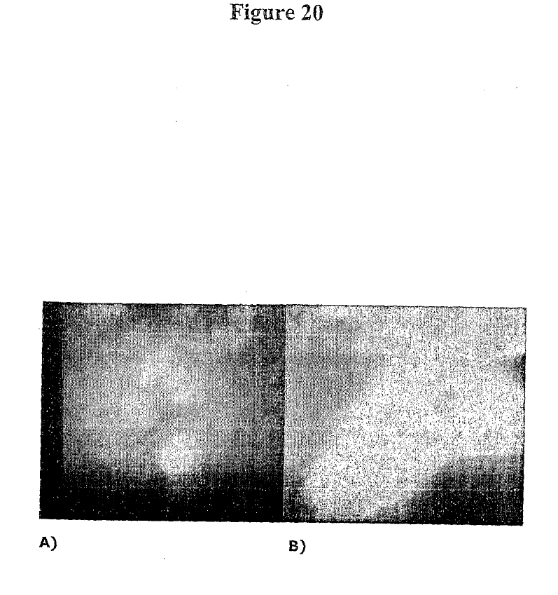

FIG. 20: Immunofluorescence staining (IF) (×400):

MCF-7 breast cancer cells were plated and consequently treated with Nicastrin RNAi for 48 h. As the half life of Nicastrin protein is 36 h, we have opted for the 48 h time point as the reference for the experiment in question. Cells were then put though the routine IF protocol, i.e. cells were washed once in PBS, fixed with 4% Formalin, permiabilized with 0.2% Triton, blocked with 10% human serum to eliminate any unspecific antibody binding, incubated for 1 h with primary Anti-E-Cadherin antibody (C-terminus, BD Transduction Laboratories Cat number: 610182) and for 1 h with anti-mouse secondary antibody. Staining demonstrates a marked up-regulation and redistribution of E-cadherin in MCF7 cells treated with NCT RNA1 (Fig B) in comparison to the non-treated control (Fig A). This result suggests that apart from inhibiting breast cancer cell proliferation, inhibition of Nicastrin may also contribute to increasing adherent potential of breast cancer cells, i.e. may induce anti-invasive properties in breast cancer cells.

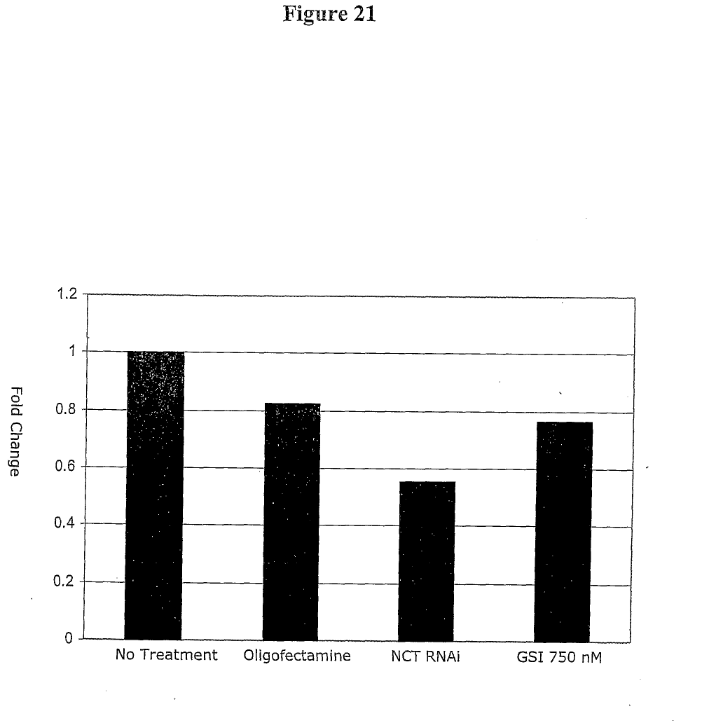

FIG. 21: Gamma-Secretase Activity Assay and CLS Reporter Assay upon NCT RNAi and GSI1 treatment:

GS Activity Assay was performed on MCF7 cell line. Cells were treated with the GSI1 at the 750 nM dose, as well as with Nicastrin RNAi at 10 nM concentration, as well as Oligofectamine as transfection treatment reagent control. The assay was performed at the 48 h time point. Results indicate that inhibiting Nicastrin may have a more profound effect in reducing Gamma secrease activity as compared to the GSI1 in the MCF7 cell line.

### EXAMPLE 1

Inhibition of GS and Reducing Expression of NCT Selectively Targets Breast Cancer Cell Lines

Summary

Results from a study conducted to determine the effect of GS inhibition on breast cancer cell lines revealed an unexpected finding. Administration of the GS peptide inhibitor GSI1 was lethal at a concentration of 1 μM to all breast cancer cell lines but was only effective against normal cell lines at concentrations in excess of 50 μM. Furthermore, knocking out the substrate binding component NCT by RNAi was found to be the most effective at selectively inhibiting cell proliferation in breast cancer cell lines. These results suggest that inhibition of GS and in particular reducing expression of NCT would be therapeutically useful in combating breast cancer and inhibiting breast cancer cell proliferation.

Results and Discussion

FIG. 6 illustrates the effect of three GSIs, DAPT, Compound E and GSI1 on proliferation in three breast cancer cell lines, MDA-MB-231 [Oestrogen receptor (ER) negative, epidermal growth factor receptor (EGFR) and HER-2 strongly positive, increasingly metastatic T47-D-MCF-MDA], T47-D [weakly ER positive, progesterone receptor positive, strongly EGFR (HER-1, cERB-B1) positive and HER-2(c-ERB-B2) positive] and MCF-7 [highly ER positive and very weakly EGFR and HER-2 positive if at all]. The breast cancer cell lines were treated with the 3 GSIs for 72 h. 20,000 cells were seeded into 24 well plates in triplicate and allowed to adhere overnight. The cells were cultured in Dulbecco's Modified Eagle's Media (DMEM)+10% foetal calf serum and 100 U/ml penicillin, 0.1 mg/ml streptomycin, 2 mM L-glutamine at 37° C. in 5% CO₂. The media was then changed to one containing different concentrations of the GSIs (50 μM, 10 μM, 1 μM, 100 nM, 10 nM and 0 M). After 72 hours the media was removed and the adherent cells removed and counted on a Coulter Counter. Graphs are plotted of the means+the standard error from the mean. In effect, the graphs show that DAPT and Compound E only have a major effect on cell numbers at 10 or 50 μM. GSI1 virtually eliminates all the cells at a concentration of 1 μM.

The effect of GSI1 on cell proliferation was investigated further in a range of breast cancer cell lines, including both oestrogen receptor positive and negative cell lines, using the Sulphorhodamine B Assay (SRB) to measure cell numbers as illustrated in FIG. 7. This shows GSI1 at different concentrations on 5 different breast cancer cell lines (MCF-7 (A), T47D (B), ZR-75-1 (C) are oestrogen receptor positive; MDA-MB-231 (D) and Cal-51 (E) are oestrogen receptor negative). 6,000 cells were plated in 6 replicates and allowed to adhere overnight. The media was replaced the following day with media containing GSI1 at different concentrations (100 nM, 250 nM, 500 nM, 750 nM, 1 μM, 2 μM, 5 μM, and 10 μM) apart from the control which was media+dimethyl sulphoxide (the solvent for GSI1 at a concentration equal to the highest in the GSI1 treated wells). One plate of cells was subjected to the Sulphorhodamine B Assay (SRB), which measures mitochondrial enzyme activity and is an accepted measure of cell numbers, on this day, day 0. Briefly, cells were fixed by adding 100 μl/well of ice-cold 40% Trichloroacetic acid to each well and leaving for 60 mins. Plates were washed 5 times in running tap water (allowing water to fill wells indirectly) and stained with 100 μl/well SRB reagent (0.4% w/v SRB [Sigma-s-9012] in 1% Acetic acid) for 30 mins. Plates were washed 5 times in 1% acetic acid and left overnight to dry before solubilizing with 100 μl/well 10 mM Tris-base. Plates were subject to shaking for 30 mins and absorbance read at 492 nm. The remaining plates were then assayed at 2-day intervals up to day 10. In each cell line the 1 μM treatment greatly reduced cell numbers and above this concentration all cells were dead. Thus, it was evident that irrespective of the cell characteristics that 1 μM and higher concentrations were an effective killing dose and doses as low as 250 nM were inhibitory to cell proliferation.

The effect of GSI1 on proliferation in normal cell lines was investigated as, illustrated in FIG. 8. This shows GSI1 at different concentrations in normal cell lines. The MCF-10A cells were treated identically as the cancer cell lines, i.e. at GSI concentrations of 100 nM, 250 nM, 500 nM, 750 nM, 1 μM, 2 μM, 5 μM, and 10 μM, however at these concentrations of GSI1 there was no effect on proliferation. Therefore we repeated in all 3 normal lines (MCF-10A, L226U19 and L226TS4) at much higher concentrations (10 μM, 20 μM, 40 μM, 60 μM and 80 μM). 10 μM GSI1 was required to kill the MCF-10A cells and over 40 μM GSI1 was required to kill the two L226 cell lines. MCF-10A cells were cultured in DMEM (Invitrogen 11995-040), 10 mg/ml Insulin (Sigma, 11882), 20 ng/ml hEGF (Trevigen, 3443-050-01), 100 ng/ml Cholera Toxin (Sigma, C8052), 500 ng/ml Hydrocortisone (Sigma, H0888), 5% Horse Serum +100U/m1 penicillin, 0.1 mg/ml streptomycin, 2 mM L-glutamine at 37° C. in 5% CO₂. L226U19 and TS4 cells were cultured in DME: F12 plus 10% FCS, 5 μg/ml insulin, 1 μg/ml Hydrocortisone, 10 ng/ml hEGF, 10 ng/ml Cholera toxin 100 U/ml penicillin, 0.1 mg/ml streptomycin, 2 mM L-glutamine at 37° C. in 5% CO₂.

In order to understand the mechanism of how GS inhibition by GSI1 leads to cell death, we investigated the effect of GS inhibition on CD44 expression which is a substrate for GS and is a cell adhesion molecule. It may be possible that if CD44 is down-regulated then cells may lift off from the tissue culture plastic and die as a result. The effect of GSI1 on CD44 expression was investigated in one cancer, ZR75-1, and one normal, MCF10A, cell line±GSI1 (at 750 nM i.e. inhibitory dose) (FIG. 9). We used an anti CD44 primary antibody and a fluorescently labelled secondary antibody to detect the CD44. Specifically, the CD44 antibody was a FITC conjugated antibody (BD Biosciences) that was used at a 1:40 dilution. The antibody was incubated for 60 mins at room temperature after fixing in formalin. Nuclei were counterstained with DAPI. There was an up-regulation of CD44 and therefore there should have been increased cell adhesion in the presence of GSI1. Thus the inhibitor was not causing the cells to become detached from the plates and die.

We next did Annexin V staining which is a marker of apoptosis. Cells were treated with GSI1 for 0, 7, 24 and 48 hours. After trypsinizing, the cells were washed with PBS 5 times and then treated with RNAase (100 μg/ml) for 20 mins then propidium iodide was added at 50 μg/ml per 1×10⁷ cells, and incubated overnight at 4° C. The samples were then filtered through a 70 μm mesh and run on a FACS with the following settings: Forward scatter 113, side scatter 356 and PE setting 380. Although there was an increase in staining indicating apoptosis, this was only approximately 10%. For example, in MDA-MB-231 cells there was a 5% increase in Annexin V staining after 7 hours of treatment and in T47D and MCF-7 cells there was an 8% increase in Annexin V staining. We have repeated these experiments with full cell cycle analysis at 7, 24 and 48 h post treatment and again detect apoptosis but at much higher levels >25%. This was using a higher concentration of GSI and the majority of cells were in G2/M Cell cycle arrest. Nevertheless it is likely that cell proliferation is inhibited via another mechanism as opposed to the induction of cellular death.

FIG. 10 illustrates the effect of GSI1 on the protein levels of the GS components and of Notch 1, Actin and Lamin A/C in a single breast cancer cell line. Shown, are Western blots of the proteins in reponse to treatment with GSI1 (750 nM) or DMSO control. All antibodies were from Abcam unless otherwise stated. Notch1 (Mouse) 1:500, Nicastrin rabbit 1:500, Pen-2 rabbit 1:1000, Presenilin, Goat 1:200, APH-1A Rabbit, 1:500, APH-1B Rabbit, 1:1000, Actin Mouse, 1:20,000(Santa Cruz), Tubulin, Mouse, 1:1000 (Sigma), Lamin NC 1:5000 (Santa Cruz). Secondary Antibodies used were goat anti mouse horseradish peroxidase (Sigma) 1:5000, goat anti-rabbit horseradish peroxidase (Sigma 1:10000) and donkey anti-goat horseradish peroxidase (Sigma 1:5000). Although GSI1 had some effects on protein expression these were marginal apart from Notch 1.

FIG. 11 illustrates the effect of knocking out Notch1 by Notch1 specific RNAi in two breast cancer cell lines: MCF-7 and MDA-MB-231. 6000 cells were plated with 6 replicates and allowed to adhere overnight. The media was changed the following day in the control cells. The other wells were treated with oligofectamine only (the transfection reagent used to punch holes in the cell walls so the RNAi enters the cells; transfection control), with oligofectamine and a non-specific RNAi from a bacterial sequence (RNAi control), with oligofectamine and an RNAi sequence for Glyceraldehyde-3-phosphate dehydrogenase (GAPDH, this is the first enzyme in the glycolysis cycle and without this the cells would shut down; positive control) and with oligofectamine and an RNAi specific for Notch1 (Notch1). As above, one plate was assayed using SRB at day 0 and others were assayed at 2-day intervals. As expected, the RNAi for GAPDH eliminated proliferation, however, although the RNAi for Notch reduced proliferation it was no more effective than the bacterial RNAi indicating that Notch1 was not involved in the effect on proliferation.

To investigate the effects on cell proliferation of knocking out GS components, RNAi was performed as above but using RNAi specific for APH-1A, Presenilin1, PEN-2 and nicastrin in 4 breast cancer cell lines (MCF-7 (A), MDA-MB-231 (B), ZR-71-1 (C) and T47D (D) cell lines) and in 1 normal cell line (MCF10A (E)) (FIG. 12). In the 4 breast cancer cell lines only nicastrin had an effect on proliferation that was markedly different from the scrambled RNAi and was analogous or close to the GAPDH. In the MCF-10A cells nicastrin had no more effect than any other RNAi.

The growth assays following RNAi were conducted as follows:

Reagents:

OPTIMEM serum free medium—Invitrogen, cat no: 31985

Silencer™ GAPDH siRNA 5 nmol siRNA+2 nmol control—Ambion, cat no: 4605

Oligofectamine Invitrogen

Method:

a) Assay layout:

Each 96 well plate uses six replicate wells, arranged in columns, for each of the following:
i) Control, untransfected cells
ii) Cells treated with transfection reagent only (Oligofectamine)
iii) Cells treated with siRNA for a control sequence not encoded in the human genome (control provided with the Silencer™ GAPDH siRNA kit)
iv) Cells treated with siRNA for GAPDH (provided with the Silencer™ GAPDH siRNA kit)
v) Cells treated with siRNA for Notch-1/NCSTN etc.

Each plate will have seven columns that can be used to assay the activity of additional siRNA's. For siRNA assays, we usually set up duplicate plates to be analysed 96 and 120 hours after transfection.

b) siRNA transfection

Day 1
1. Harvest cells from an exponentially growing culture. A good single cell suspension is needed for seeding.
2. Seed 6×10³ cells per well of a flat-bottomed 96-well tissue culture plate in full medium (Phenol red supplemented DMEM/10% FCS, GPS).
3. Allow cells to adhere and spread over a 24-hour period.

Day 2
1. Gently aspirate off medium from each well and wash once with 100 μl of OPTIMEM.
2. Remove and replace medium in each well with another 100 μl of OPTIMEM
3. Set aside plates in the hood at room temperature.
4. For each set of transfections (6 wells) set up the following in a sterile 1.5 ml microfuge tube:
   18.2 μl of OPTIMEM
   2.8 μl of Oligofectamine (taken from a stock that has been briefly vortexed just before use)
5. Vortex vigorously and incubate for 15 minutes at room temperature.
6. During the Oligofectamine incubation, dilute 2.1 μl of siRNA (stock at 20 μM) into 116.9 μl of OPTIMEM in a sterile 1.5 ml microfuge tube:
7. On completion of step 5, add the diluted siRNA (119 μl) to the Oligofectamine suspension and gently mix by tituration (DO NOT VORTEX).
8. Incubate for 15 minutes at room temperature.
9. Add 20 μl of the siRNA transfection mix to each well to be assayed.
10. Once addition of the transfection mixes is complete, incubate the plate in the TC incubator under normal culture conditions for 4 hours.
11. Add 100 μl of Phenol red supplemented DMEM/20% FCS, GPS to each well and return plates to the TC incubator for growth.
12. Measure cell growth by the SRB assay

The effects of knocking out the GS components by RNAi on gene expression of the GS components and its substrates were also analysed in two breast cancer cell lines (MCF-7, Table 2; MDA-MB-231, Table 3), using reverse transcriptase polymerase chain reaction (RT-PCR). In the gene column are the genes knocked out by RNAi and the other columns are the effect of this knockout on other genes. When each RNAi was performed, it knocked out the target gene but when nicastrin was knocked out it not only knocked out nicastrin but also knocked down (reduced not eliminated) the genes for the other GS components and increased the expression of Notch 2 which is a potential tumour suppressor gene. These experiments were done with 2 different RNAi products from Dharmacon (both standard RNAi and also the more specific On-Target™ RNAi).

TABLE 2
Effect of RNAi on gene expression in MCF-7 cells

| | | Down | |
|---|---|---|---|
| Gene | Up regulation | Regulation | No effect |
| Nicastrin | None | Nicastrin, Psen1, | APH1A |
| | | Psen 2, CD44, Jagged | Pen2 |
| | | 1, Jagged2 | |
| APH1A | Nicastrin, Psen1, Psen2, | APH1A | Jagged 2 |
| | Pen2, Jagged1, | | |
| | CD44 | | |
| Psen1 | Nicastrin, APH1A, | Psen1 | Jagged 2 |
| | Psen2, Pen 2, | | |
| | Jagged1, CD44 | | |
| PEN 2 | Nicastrin, | PEN2 | None |
| | APH1a, Psen1, | | |
| | Psen2, CD44, | | |
| | Jagged 1, Jagged2 | | |

TABLE 3
Effect of RNAi on gene expression in MDA-MB-231 cells

| | | Down | |
|---|---|---|---|
| Gene | Up regulation | Regulation | No effect |
| Nicastrin | Notch 2 | Nicastrin, PEN2, | Notch1, 3, APH1B, |
| | | Psen1, APH1A | Jagged 1 |
| APH1A | Notch 2, Nicastrin, | APH1A | Notch1, 3, APH1B, |
| | Jagged 1 | | PEN2, Psen |
| PEN 2 | Notch 2, Nicastrin, | PEN2 | Notch1, 3 |
| | APH1A, APH1B | | |
| | Psen1, Jagged 1 | | |
| Psen | | Nicastrin, PEN2, | Notch1, 2, 3, |
| | | Psen1, Jagged1 | APH1A APH1B, |

All PCRs were run with a 96° C. Hotstart for 10 mins and a denaturation at 96° C. for 30 secs and elongation at 72° C.

The following are the primer sequences used (forward then reverse), the size of the product, the annealing temperature, the time and the number of cycles:

Psen2,
PSen2F-CTGCCCAGGAGAGAAATGAG,
Psen2 CAGTCAAGGGAGGCTCAAAG
Size 198nt , 61° C., 25 sec, 30 cycles
Jagged-1
JG1F CTCCTGTCGGGATTTGGTTA,
JG1R CCACAGACGTTGGAGGAAAT,
Size 414nt 58° C., 30 sec, 30 cycles
Jagged-2
JG2F GTCAAGGTGGAGACGGTTGT,
JG2R ATCCTCGTCCTCCTCATCCT,
Size 345nt, 60° C., 30 sec, 32 cycles
Notch-1
NT1-F GCAACAGCTCCTTCCACTTC,
NT1R CCACGAAGAACAGAAGCACA,
SIZE 495nt, 59° C., 27 sec, 35 cycles
Notch-2
NT-2F ATGACTGCCCTAACCACAGG,
NT-2R CTGGAGTACAGGAGGCGAAG,
Size 264nt, 59° C., 20 sec, 30 cycles
Notch-3
NT-3F GTGTGTGTCAATGGCTGGAC,
NT-3R CGATAGAGCACTCGTCCACA,
Size 292nt, 56° C., 20 sec, 32 cycles
Notch-4
NT-4F GGCTTCTACTCCGCTTCCTT
NT-4R CAACTTCTGCCTTTGGCTTC
Size 345nt, 57° C., 25 sec, 30 cycles
PSEN
PSEN-F GGTCCACTTCGTATGCTGGT
PSEN-R GCGAGGATACTGCTGGAAAG
SIZE 314 bp, 60° C., 15 sec, 30 cycles
PEN-2
PEN-2-F TTCCTTGTCCCAGCCTACAC
PEN-2-R GGTCCTTTATTGGGGGATGT
SIZE 329 bp, 61° C., 25 sec, 30 cycles
APH-1A
APH-1-F CCGCTTTGCCTACTACAAGC
APH-1-R CCAAAAGGTATGGAGCAGGA
SIZE 265 bp, 58° C., 20 sec, 32 cycles
Nicastrin,
NicF-CAAAGCACCTTCAGCATCAA,
NicR CGAGCTGCCAATGTAGTCAA,
Size 315nt, 58° C., 25 sec, 32 cycles.

Conclusion

These results show that inhibition of GSI by GSI1, selectively kills breast cancer cell lines at concentrations of 1 μM GSI1, but concentrations in excess of 50 μM GSI1 are required to have any effect on normal cell lines. Knocking out the substrate binding component of the GS complex, nicastrin, by RNAi was also found to be the most effective at selectively inhibiting cell proliferation in breast cancer cell lines, with nicastrin RNAi having no more effect than any RNAi in normal cells. Furthermore, knocking out nicastrin by RNAi, not only reduced expression of nicastrin but also of the other GS complex components. Together, these results suggest that inhibition of GS and in particular reducing expression of NCT would be therapeutically useful in combating breast cancer and inhibiting breast cancer cell proliferation.

### EXAMPLE 2

NCT is Correlated with Metastatic Potential of Breast Cancer

Summary

Staining of normal and cancerous breast tissue from the complete range of breast cancer patients in terms of hormone and Her-2 expression revealed a significant correlation between NCT expression and metastatic potential, suggesting that NCT would be useful as a biomarker for breast cancer.

Results and Discussion

FIG. 13 illustrates nicastrin expression in 3 normal (TS4, LU19 and MCF10A) and 3 breast cancer (ZR75, MDA-231 and T47D) cell lines. When we stained normal and cancer cell lines for nicastrin, there were differences in expression between the cell lines but these were not major. These results have been confirmed using an immunogenic peptide to which the antibody against nicastrin was raised to confirm that this staining is nicastrin. Most of the cells appear to have the nicastrin in the nucleus as shown by the DAPI stained blue. However, cell lines may not be representative of breast cancer cells in vivo. We believe that in cell culture there are fewer substrates of GS present and therefore nicastrin may not be required at the high levels seen in individuals.

The Notchs are believed to of great importance in so-called breast cancer stem cells, as defined by those cells which express epithelial cell adhesion molecule (Ep-CAM) positive, CD44 positive and CD24 low/negative Ep-CAM⁺/CD44⁺/CD24^{low/-}. When these cells were purified from fresh breast tumour and compared to the Ep-CAM⁺/CD44⁺/CD24⁺ cells, which can be defined as benign breast cancer or non-stem cell breast cancer cells, there is differential expression of nicastrin in that in the benign cells it is expressed in the nucleus with a little in the cytoplasm whereas in the stem cells it is predominantly in the cytoplasm which is where it will be active (FIG. 14). The cells were purified as follows. Breast tissue was minced for 1-5 hours at 37° C. with type IA collagenase (1 mg/ml) in DMEM plus 5% foetal calf serum (FCS) and 2 mM L-glutamine, 100 U/ml penicillin, 0.1 mg/ml streptomycin, 50 U/ml polymixin B, 2.5 mg/ml amphotericin B. Undigested material was removed using a 50 μm pore nylon mesh. Tissue remaining on the mesh was subjected to further collagenase digestion (1-3 hours) and again filtered through a 50 μm pore nylon mesh. The two filtrates were refiltered with progressively smaller filter sizes (50 μm to 28 μm). Under these digestion conditions the majority of normal epithelial cells remained in intact organoids or as clumps and were removed by the filtration steps. Epithelial cells were immunoaffinity purified using superparamagnetic, polystyrene beads (Dynal Ltd., New Ferry, Wirral, UK) coated with a mouse IgG1 monoclonal antibody (MAb Ber-EP4) specific for two (34 and 39 kDa) glycopolypeptide membrane antigens. These cells were then further purified using antibodies against CD44-Phycoerythrin labelled and CD24-FITC labelled (BD Biosciences). Initially incubated with anti-CD44 for 1 hour at 4° C. then incubated with anti-PE microbeads (Miltenyi Biotech) the positive cells from this sort were then incubated with anti-CD24 antibody for 1 hour at 4° C. then incubated with anti-FITC microbeads (Miltenyi Biotech). The cells positive for CD24 were designated to be ‘benign’ breast cancer cells and the CD24 negative the breast cancer stem cells. Cells were either pelleted and stored at −70° C. for RNA extraction, or cytocentrifuged onto slides for staining.

We stained breast cancer and normal tissue for nicastrin (using the brain as a positive control) as illustrated in FIG. 15. 5 μm sections were cut from paraffin embedded tissue and stained using a polyclonal anti nicastrin antibody at a dilution of 1:500 (Sigma) and the secondary antibody used was goat anti rabbit horse radish peroxidase at 1:5000 dilution (sigma). The tissues in the slide were Alzheimer's brain tissue, normal breast from reduction mammoplasty, a breast tumour (ER,PR and HER-2 positive) and from a lymph node from the same patient. In normal breast, nicastrin is restricted to the luminal epithelial cells (the cells that become malignant in over 95% of all breast cancers) and is weakly expressed. In breast cancer it is also restricted to the luminal epithelial cells but at much higher levels. In the lymph nodes, nicastrin is only expressed in the invading tumour cells not in the normal lymph nodes.

We have to date stained 19 normal and 65 breast cancer tissues from the complete range of breast cancer patients in terms of hormone and Her-2 expression. Results show that over 90% of breast cancer tissues were nicastrin positive, all ER/PR/Her-2 positive tissue was NCT positive, as were the triple negatives. The highest nicastrin expression was evident in the HER2 positive patients. In the normal tissue 50% stained positive for nicastrin, however, this was only weak.

Conclusion

Although nicastrin is in normal breast tissue it is only weakly expressed and is specific to the luminal epithelial cells i.e. no stromal or myoepithelial cells stain positive. Nicastrin expression is markedly higher in breast cancer tissue, it is also restricted to the luminal epithelial cells. As yet no specific correlations are evident with known prognostic indicators such as oestrogen and progesterone receptor or Her-2 although in this limited study the highest nicastrin staining (+++) was seen in the tissue highly positive for HER-2.

### EXAMPLE 3

Treatment of Breast Cancer Cells with MAPK/ERK Inhibitor U0126 GS Activity

Summary

Studies of the effect of a MAPK/ERK inhibitor on GS activity in breast cancer cells revealed a 9-fold increase in GS activity. Since some of the novel growth factor receptor targeted therapies in breast cancer act via MAPK/ERK inhibition, consequent increase in GS activity resulting in cell proliferation is expected to represent a novel treatment resistant pathway. Therefore, targeting the GS complex via NCT is likely to restore sensitivity to these therapies and further contribute to the anti-proliferative effects of these drugs.

Results and Discussion

The effects of the ERK1/2 inhibitor U0126 and other anti-proliferative agents on GS activity were investigated (FIG. 16). Cancer and normal cell lines were treated with oestrogen (E2), faslodex (FAS), Gefitinib (Gef 15 μM), GSI1, and the ERK1/2 inhibitors U0126 (U) (10 μM) and PD98059 (PD) (10 μM) for 48 hours and a fluorescent assay performed to measure GS activity. The figures show relative fluorescence therefore the amount of fluorescence in the treated cells relative to the untreated minus background (these figures are corrected for background). Although, as can be seen from the staining of MCF-7 cells, all but the Faslodex appear to increase notch intracellular domain expression localized to the nucleus (as a surrogate marker of GS activity) compared to the untreated cells (NO), the increase in the GS activity when measured using the assay was only markedly increased by U0126 (6-12 fold). In MCF-10A cells the GS activity was increased approximately 4 fold. We believe that active ERK1/2 phosphorylates nicastrin and attenuates GS activity, while ERK1/2 inhibition elevates GS activity. We postulate that this provides a mechanism by which breast cancer cells can become resistant to growth factor receptor inhibitors. Targeting GS complex via nicastrin may restore sensitivity to growth factor receptor inhibitors and thus further contribute to the anti-proliferative effects of these drugs.

Conclusion

These results demonstrate that treatment of breast cancer cell lines with the ERK1/2 inhibitor U0126 increases the activity of the GS complex. This is a possible mechanism by which breast cancer cells become resistant to growth factor receptor inhibitors. Therefore, targeting the GS complex via nicastrin may restore sensitivity to inhibitors of the MAPK/ERK pathway and further contribute to the anti-proliferative effects of these drugs.

### EXAMPLE 4

### Nicastrin Staining in Breast Tissue (FIG. 17)

We have used 126 archival paraffin tissue blocks of breast carcinoma as well as 40 cases of paraffin embedded normal breast tissue from reduction mammoplasty and 10 benign breast lesions to stain for NCT, according to my developed IHC protocol. NCT staining was detected in 69.5% (89/126) of breast cancer cases, suggesting that it may act as an oncogene in breast cancer development and progression. (FIG. 5A). Nicastrin is localized in the cytoplasm in breast cancer tissue and its expression can be scored as (1+/2+/3+). Preincubation with the Immunizing peptide eliminated any expression of the protein of interest, confirming the specificity of the anti-NCT antibody I was using (FIG. 5D). My results indicate that NCT is not expressed or is weakly expressed in normal breast tissue (FIG. 5C). It is also absent in the adjacent normal tissue of breast cancers that express Nicastrin (FIG. 5F). NCT is strongly expressed in the myoepithelial cell layer of benign breast lesions (fibroadenoma). This suggests a possible myoepithelial-to-luminal switch in expression when progressing from benign via precancerous lesions to breast cancer. NCT expression is preserved in the positive lymph nodes of the corresponding positive breast carcinoma (FIG. 5E). The specimens also confirm NCT enrichment in the muscle layer of an arterial blood vessel (FIG. 5C-arrow), as previously reported.

Statistical analyses (Pearson chi² and Fisher's Exact Test) were used to correlate NCT expression to that of the ER, PR and HER2 receptor. There was an association between NCT and ER (p=0.01) and NCT and PR (p=0.025). Out of 89 cases that were positive for NCT, 55.1% were also positive for ER (49/89). In the group of NCT negative cases (37), only 11 (29.7%) were ER positive. There was also an association between NCT and HER2 (p=0.008). Thirty-nine cases out of 89 positive for NCT, were also positive for HER2 (43.8%), while in the group of 37 NCT negative cases, only 7 (18.9%) were HER2 positive. There was strong evidence of association (p=0.002) between NCT and ER/HER2 status (combined into a single categorical variable). More than 72% of ER⁺HER2⁺ cases were positive for NCT, 87% of ER⁻HER2⁺ cases were NCT positive and 50% of the triple negative cases showed NCT expression.

### EXAMPLE 5

### Gamma-Secretase Activity Assay and CSL Reporter Assay Upon GSI1 Treatment (FIG. 19)

a) Gamma-Secretase Activity Assay:

1.5×106 cells were plated out and allowed to adhere overnight. The next day, cells were treated with increasing concentrations of GSI1 (750 nM, 2 uM, 5 uM) for 24 and 48 hours. We used the R&D Systems Gamma Secretase Activity Kit (as previously reported- ref from R&D Spec sheet) to determine Gamma Secretase Activity in breast cancer cell lines. Cells were washed twice with ice-cold PBS and all following steps, according to manufacturer's protocol were performed at 4 degrees. Briefly, cells were harvested in the Cell Extraction buffer, and incubated on ice for at least 10 minutes, to allow for maximal Buffer activity. Lysates were centrifuged at 10,000×g for 1 minute. Supernatants were collected with a total protein yield between 0.5-1 mg/ml. Pierce BCA Protein Assay was used to determine protein concentration in each sample. 200 microg of was used and incubated with the gamma-secretase fluorogenic substrate for 2 hours at 37° C. Fluorescence was measured at 355/460 nm.

Results:

Reduction in gamma-secretase activity after treatment with GSI1 was dose dependant and already significant at the dose of 750 nM.

b) CSL Reporter Assay:

For the Luciferase based reporter assay, 5×104 cells were plated in 48 well plates and allowed to adhere over night. The following day, cells were transfected with 200 ng of the 23A plasmid (Hes-1 reporter). 24 h after transfection, transfection media was evacuated and replaced with treatment media (750 nM, 2 microM, 5 microM of the GSI1 and a vehicle treated control). Cells were incubated for 24 h at 37 C. At the 24 h time point the Dual Glow (Promega) Luciferase Kit was implemented according to manufacturer's protocol).

Results:

Transcriptional activity of Hes1, as one of the Notch target genes, measured by reflected Luciferase activity, was affected by GSI1 treatment. GSI1 treatment of cells that were transfected with the Heslwt-Luc (23A) reporter construct, resulted in a dose dependant decrease of the reporter activity at 24 h

### EXAMPLE 6

### Effect of Nicastrin RNAi on Breast Cancel Cell Morphology and Motility

MCF-7, BT474, SKBR3 and MDA-MB231 breast cancer cells were plated and consequently treated with Nicastrin RNAi. Cells were exposed to live imaging by transmitted light microscopy for 12 h continuously starting at hour 24 post transfection, with images being taken in 12 minute intervals throughout. Images indicate that inhibition of Nicastrin in the invasive and highly metastatic cell line MDA-MB231, causes a change in cell shape, converting a spindle like morphology to a more rounded and ‘normal-like’ cell phenotype. Also, inhibition of Nicastrin reduces motility in MDA-MB231 cell line. This result indicates that inhibiting Nicastrin may reduce the invasive and metastatic potential of breast cancer cells.

Summary

We consider that inhibiting Nicastrin in breast cancer cell lines causes significant reduction of cell proliferation while having very little effect on normal breast cell lines. Nicastrin is not expressed or is weakly expressed in normal breast tissue and it is upregulated in breast cancer, suggesting its oncogenic role. Inhibiting Nicastrin in breast cancer cell line MCF7 also causes upregulation of a cell-to-cell adhesion protein E-cadherin suggesting that Nicastrin inhibitoin may strengthen cell-to-cell junctions, slowing down motility of breast cancer cells and causing then to acquire a less malignant and invasive phenotype.

### REFERENCES

Al-Hajj M, Wicha M S, Benito-Hernandez A, Morrison S J, Clarke M F. Prospective identification of tumorigenic breast cancer cells. Cell Biol 2003; 10:3983-3988.
Auvinen P, Tammi R, Johansson R, Kosma V M. Expression of CD44s, CD44v3and CD44v6 in benign and malignant breast lesions: correlation and colocalization with hyaluronan. Histopathology 2005; 47:420-428
Bankfalvi A, Terpe H J, Breukelmann D, Bier B, Rempe D, Pschadka G, Krech R, Lelle R J, Boecker W. Immunophenotypic and prognostic analysis of E-cadherin and beta-catenin expression during breast carcinogenesis and tumor progression: a comparative study with CD44. Histopathology 1999; 34:25-34
Callahan R, Egan S E. Notch signaling in mammary development and oncogenesis. Journal of Mammary Gland Cell Biology 2004; 9(2):145-158.
Capell. A, Beher D, Prokop S, Steiner H, Kaether C, Shearman M S, Haass C. γ-secretase complex assembly within the early secretory pathway. J Biol Chem 2005; 280:6471-6478.
Chen Y, Fischer W H, Gill G N. Regulation of the ERBB-2 promoter by RBPJk and Notch. J Biol Chem 1997; 272(22):14110-14114
Chodosh L A, Boxer R B, Moody S E, Keister B A, Sarkisian C J, Jang J W, Belka G K, Blakely C M, Portocarero C P, Sterner C J, Notarfrancesco K L and Kauh E A. Breat Cancer Res 2003: 5(Suppl 1):5.
De Strooper B. Nicastrin: gatekeeper of the gamma-secretase complex. Cell 2005; 122(3):318-320
Gotte M, Yip G W. Heparanase, Hyaluronan, and CD44 in Cancers: A Breast Carcinoma Perspective. Cancer Res 2006; 66(21):10233-10237
Gustaffson M V, Zheng X, Pereira T, Gradin K, Jin S, Lundkvist J, Ruas J L, Poellingee L, Lendahl U, Bondesson M. Hypoxia requires Notch signalling to maintain the undifferentiated cell state. Developmental Cell 2005; 9:617-628
Hainaud P, Contreres J O, Villemain A, Liu L X, Plouet J, Tobelem G, Dupuy E. The Role of the Vascular Endothelial Growth Factor-Delta-like 4 Ligand/Notch4-Ephrin B2 Cascade in Tumor Vessel Remodeling and Endothelial Cell Functions. Cancer res 2006; 66(17):8501-8510
Hu Y, Fortini M E. Different cofactor activities in gamma-secretase assembly:evidence for a nicastrin-APH1 subcomplex. J Cell Biol 2003; 161(4):685-690
Jackson A. L., Bartz S. R., Schelter J., Kobayashi S. V., Burchard J., Mao M., Li B., Cavet G., Linsley P. S. 2003. Expression profiling reveals off-target gene regulation by RNAi. Nat. Biotechnol. 21: 635-637
Junttila T T, Sundvall M, Lundin M, Lundin J, Tanner M, Harkonen P, Joensuu H, Isola J, Elenius K. Cleavable ErbB4 isoform in estrogen receptor regulated growth of breast cancer cells. Cancer res 2005; 65(4):1384-1393
Kang D E, Yoon I S, Repetto E, Busse T, Yermian N, Ie L, Koo E H. Presenilins mediate PI3K/AKT and ERK activation via select signalling receptors. J Biol Chem 2005; 280(36):31537-31547
Kilber R L and Planas-Silva M D. Cyclin D1 is necessary for tumoxifen-included cell cycle progression in human breast cancer cells. Cancer Res. 2006; 66(23): 11478-84
Kim S K, Park H J, Hong H S, Baik E J, Jung M W, Mok-Jung I. ERK1/2 is an endogenous negative regulator of the gamma-secretase activity. FASEB J 2006; 20(1)
Lee H J, Jung K M, Huang Y Z, Bennett L B, Lee J S, Mei L, Kim T W. Presenilin-dependant gamma-secretase-like intramembrane cleavage of ErbB4. J Biol Chem 2002;277(8):6318-6323
Lerwill M F. The Evolution of Lobular Neoplasia. Adv Anat Pathol 2006; 13(4):157-165
Maatta J A, Sundvall M, Junttila T T, Peri L, Laine V J, Isola J, Egeblad M, Elenius K. Proteolyitic cleavage and phosphorylation of tumor-associated ErbB4 isoform promote ligand-independent survival and cancer cell growth. Mol Biol Cell 2006; 17(1):67-79
Modureira P A, Varshochi R, Constantinidou D, Francis R E, Coombes R C, Yas K M and Lam E W. The Forkhead box MI protein regulates the transcription of the estrogen receptor alpha in breast cancer cells. J Biol Chem. 2006; 281(35): 25167-76
Marhaba R, Bourouba M, Zoller M. CD44v6 promotes proliferation by peristing activation of MAP kinases. Celluler Signalling 2005; 17:961-973
McKenzie G, Ward G, Stallwood Y, Briend E, Papadia S, Lennard A, Turner M, Champion B, Hardingham G E. Cellular Notch responsiveness is defined by phosphoinositide 3-kinase-dependent signals. BMC Cel Biol 2006; 28:7:10
Mungamuri S K, Yang X H, Thor A D, Somasundaram K. Survival signalling by notch1: Mammalian target of rapamycin (mTOR)-dependent inhibition of p53. Cancer Res 2006; 66(9):4715-4724
Nyborg A C, Ladd T B, Zwizinski C W, Lah J J, Golde T E. Sortilin, SorCSIb, nd SorLA VpsI0p sorting receptors are novel gamma-secretase substrates. Molecular. Neurodegeneration 2006; 1:3
Reedijk M, Odorcic S, Chang L, et al. High-level coexpression of JAG1 and NOTCH1 is observed in human breast cancer and is associated with poor overall survival. Cancer Res 2005; 65:8530-8537.
Russo J and Russo I H, Experimentally induced mammary tumors in rats. Breast Cancer Res Treat 1996: 39:7-20.
Shah S, Lee S-F, Tabuchi K, Hao Y H, Yu C, LaPlant Q, Sudhof T, Yu G. Nicastrin functions as a gamma-secretase substrate receptor. Cell 2005; 122:435-447
Sheridan C, Kishimoto H, Fuchs R K, Mehrotra S, Bhat-Nakshatri P, Turner C H, Goulet R Jr, Badve S, Nakshatri H. CD44+/CD24− breast cancer cells exhibit enhanced invasive properties, an early step necessary for metastasis. Breast Cancer Res 2006; 8(5):R59
Soares R, Balogh G, Guo S, gartner F, Russo J, Schmitt F. Evidence for the notch signalling pathway on the role of estrogen in angiogenesis. Mol Endocrinol 2004; 18(9):2333-2343
Srinivasan R, Gillett C E, Barnes D M, Gullick W J. Nuclear expression of the c-erb-4/HER-4 growth factor receptor in invasive breast cancer. Cancer Res 2000; 60(6):1483-1487
Stylianou S, Clarke R B, Brennan K. Abberant Activation of Notch Signaling in Human Breast Cancer. Cancer Res 2006; 66:(3)
Sun Y, Lowther W, Kato K, Bianco C, Kenney N, Strizzi L, Raafat D, Hirota M, Khan N I, Sharon B, Jones B, Salomon D, Callahan R. Notch4 intracellular domain binding Smad3 and inhibition of the EGF-beta signalling. Oncogene 2005; 24:5365-5374
Walker E S, Martinez M, Wang J, Goate A. Conserved residues in juctamembrane region of the extracellular domain of nicastrin are essential for gamma0secretase complex formation. Journal of Neurochemistry 2006; 98:300-309
Weng A P, Miliholland J M, Yashiro-Ohtani Y, Arcangeli M L, Lau A, Wai C, del bianco C, Rodriguez C G, Sai H, Tobias J, Li Y, Wolfe M S, Shachaf C, Felsher D, Blacklow S C, Pear W S, Aster J C. C-Myc is an important direct target of Notch1 in T-cell aculet Lymphoblastic leukaemia/lymphoma. Genes&development 2006; 20:2096-2109
Zhu Y, Sullivan L L, Nair S S, Williams C C, Pandey A K, Marrero L, Vadlamudi R K, Jones F E. Coregulation of Estrogen Receptor by ERBB4/HER4 Establishes a Growth-promoting Autocrine Signal in Breast Tumor Cells. Cancer Res 2006; 66(16)

1. A method of combating breast cancer in a mammalian individual, the method comprising administering to the individual an agent which reduces the function of the gamma secretase (GS) complex.

2. (canceled)

3. (canceled)

4. (canceled)

5. (canceled)

6. (canceled)

7. (canceled)

8. A method of claim 1 wherein the agent reduces the expression of nicastrin.

9. A method according to claim 8, wherein the agent is a nicastrin siRNA molecule, a nicastrin antisense oligonucleotide or a nicastrin specific ribozyme.

10. (canceled)

11. A composition for combating breast cancer in a mammalian individual comprising an agent selected from the group consisting of (i) an agent which binds to the GS complex substrate binding site; (ii) an agent which induces an inhibitory conformational change in the GS complex; (iii) an agent which modifies the active site of the GS complex; (iv) a neutralising antibody against the GS complex or a neutralising antibody against one of its components nicastrin, presenilin, presenilin enhancer-2 and anterior pharynx defective-1; and (v) an agent which sequesters the enzyme preventing its interaction with its substrate.

12. A composition of claim 11 wherein the agent comprises the GS inhibitor GSI1 (-Z-Leu-Leu-Nle-CHO).

13. A method according to claim 1 wherein the individual is administered an additional anti-cancer agent.

14. (canceled)

15. (canceled)

16. (canceled)

17. (canceled)

18. (canceled)

19. (canceled)

20. A method of claim 13 wherein the additional anti-cancer agent is an inhibitor of the MAPK/ERK signalling pathway.

21. A method according to claim 20 wherein the inhibitor of the MAPK/ERK signalling pathway is the ERK1/2 inhibitor U0126.

22. (canceled)

23. A composition comprising an agent of claim 11 and an additional anti-cancer agent.

24. (canceled)

25. A composition according to claim 23 wherein the additional anti-cancer agent is an inhibitor of the MAPK/ERK signalling pathway.

26. A kit of parts comprising:
an agent which reduces the function of the GS complex and
an additional anti-cancer agent

27. (canceled)

28. (canceled)

29. A method of assessing an individual's risk of developing breast cancer; or of assessing the progression of breast cancer in an individual; or for assisting in the diagnosis of breast cancer in a mammalian individual, comprising:
a) providing a biological sample from the individual, and
b) determining one or more of:
i. amount and/or function of nicastrin
ii. amount of nucleic acid encoding nicastrin
iii. cellular location of nicastrin.

30. (canceled)

31. (canceled)

32. An agent which is capable of being used in determining one or more of: (i) the amount and/or function of nicastrin; (ii) the amount of nucleic acid encoding nicastrin;
and (iii) the cellular location of nicastrin, for use in assessing an individual's risk of developing breast cancer; or for assessing the progression of breast cancer in an individual; or for assisting in the diagnosis of breast cancer in an individual.

33. A method of identifying a compound which may be useful in the treatment of breast cancer, comprising determining whether a test compound modulates at least one of (i) the amount and/or function of nicastrin, (ii) the amount of nucleic acid encoding nicastrin and (iii) the cellular location of nicastrin.

34. A method according to claim 33, further comprising:
selecting a test compound which modulates at least one of (i) the amount and/or function of nicastrin, (ii) the amount of nucleic acid encoding nicastrin and (iii) the cellular location of nicastrin;
modifying the test compound; and
testing the ability of the modified compound to modulate at least one of (i) the amount and/or function of nicastrin, (ii) the amount of nucleic acid encoding nicastrin and (iii) the cellular location of nicastrin.

35. A method according to claim 34 wherein the method is performed in vitro.

36. A method according to claim 34 wherein the method is performed in vivo.
